(19) United States
(12) Patent Application Publication
(10) Pub. No.: US 2023/0020448 A1
(43) Pub. Date: Jan. 19, 2023

(54) NEEDLING DEVICES AND PENETRATION DEPTHS

(71) Applicant: Follica, Inc., Boston, MA (US)

(72) Inventors: David CHASTAIN, Boston, MA (US); Rajan PATEL, Boston, MA (US); Jason Venkat BHARDWAJ, Newton, MA (US)

(21) Appl. No.: 17/782,327

(22) PCT Filed: Dec. 4, 2020
(86) PCT No.: PCT/US2020/063194

§ 371 Date: Jun. 3, 2022

Related U.S. Application Data
(60) Provisional application No. 62/944,232, filed on Dec. 5, 2019.

(51) Int. Cl. A61B 17/34

(52) U.S. Cl. CPC A61B 17/3476; A61B 17/3494; A61B 2017/00752

(57) ABSTRACT

A needling device includes a plurality of needles forming a needle array, and a motor assembly for driving the needle array, where each of the plurality of needles includes a needle tip at one end, and tapers at a taper angle and along a taper length to a maximum needle diameter at the other end, and in use, a skin reference surface of the needling device is in contact with a subject's skin.

### 1. CROSS-REFERENCE TO RELATED APPLICATION

This application claims the benefit of priority to U.S. Provisional Patent Application No. 62/944,232, filed Dec. 5, 2019, which is hereby incorporated by reference in its entirety for all purposes.

### 2. BACKGROUND OF THE INVENTION

### 2.1 Field of the Invention

The following description relates to a needling device for needling of a subject's skin by a user such as a physician or any user. In certain embodiments, the subject is in need of inducing hair growth or hair follicle neogenesis, or is in need of preventing hair loss. For example, a needling device may be applied to a subject's skin for hair growth applications, or may also be used for wrinkle reduction, scar revision, hair removal, tattoo removal, and pigmentation.

### 2.2 Description of Related Art

Needling devices are typically used for tattoo removal or wrinkle reduction mechanisms by lightly penetrating a subject's skin and without penetrating deeper areas of a subject's skin or scalp. Needling devices have also been used for hair growth applications. However, conventional needling devices do not allow a user to easily achieve optimal therapeutic depth and puncture precision in these various treatments and procedures. Conventional needling devices fail to provide optimal needle dimensions, needle orientations, skin reference dimensions, motors, motor linkages, among other features, that allow optimal therapeutic depth and puncture precision.

### 3. SUMMARY

In an aspect, a needling device includes a plurality of needles forming a needle array, and a motor assembly for driving the needle array.

Each of the plurality of needles may include a needle tip at one end, and may taper at a taper angle and along a taper length to a maximum needle diameter at the other end, and the maximum needle diameter may range from approximately 0.20 mm to approximately 0.24 mm.

The taper length may range from approximately 1 mm to approximately 2 mm.

The taper angle may range from approximately 5 degrees to approximately 15 degrees.

The needle tip may have a tip radius ranging from approximately 0.015 mm to approximately 0.025 mm.

In use, a skin reference surface of the needling device may be in contact with a subject's skin, and the skin reference surface may have a surface area ranging from approximately 45 mm² to approximately 105 mm².

In use, a skin reference surface of the needling device may be in contact with a subject's skin, and an average distance between each needle of the plurality of needles of the needle array and the skin reference surface may range from approximately 0.10 mm to approximately 2.5 mm.

A distance between each needle of the plurality of needles of the needle array and the skin reference surface may be the same for all needles.

A first distance between one of the plurality of needles of the needle array and the skin reference surface may be different than a second distance between another of the plurality of needles of the needle array and the skin reference surface.

The motor assembly may include a motor linkage, and the motor linkage may include a rotational component with a total mass ranging from approximately 0.5 grams to approximately 35 grams and a rotational radius ranging from approximately 1.5 mm to approximately 3.5 mm, and the motor linkage may include a linear component with a total mass ranging from approximately 1.5 grams to approximately 3.5 grams.

An actual penetration depth of the plurality of needles into a subject's skin may not exceed a depth setting on the needling device.

A mean value of an actual penetration depth of the plurality of needles may be at least 0.2 mm in response to a depth setting of the needling device of 0.5 mm, at least 0.6 mm in response to a depth setting of the needling device of 1.5 mm, and at least 0.75 mm in response to a depth setting of the needling device of 2.0 mm.

In use, an actual penetration depth of the plurality of needles may be at least 50% of a target depth based on a depth setting of the device for at least 45% of all needle strikes of the plurality of needles.

In use, an actual penetration depth of the plurality of needles may be at least 50% of a target depth based on a depth setting of the device for at least 35% of all needle strikes of the plurality of needles.

In use, an actual penetration depth of the plurality of needles may be at least 50% of a target depth based on a depth setting of the device for at least 25% of all needle strikes of the plurality of needles.

In use, an actual penetration depth of the plurality of needles may be at least 50% of a target depth based on a depth setting of the device for at least 15% of all needle strikes of the plurality of needles.

The needling device may further include a sheath assembly comprising the needle array and a main unit comprising the motor assembly.

In another aspect, a needling device includes a plurality of needles forming a needle array, and a motor assembly for driving the needle array, wherein each of the plurality of needles includes a needle tip at one end, and tapers at a taper angle and along a taper length to a maximum needle diameter at the other end, and the maximum needle diameter ranges from approximately 0.20 mm to approximately 0.24 mm.

In another aspect, a needling device includes a plurality of needles forming a needle array, and a motor assembly for driving the needle array, wherein each of the plurality of needles includes a needle tip at one end, and tapers at a taper angle and along a taper length to a maximum needle diameter at the other end, and wherein the taper length ranges from approximately 1 mm to approximately 2 mm.

In another aspect, a needling device includes a plurality of needles forming a needle array, and a motor assembly for driving the needle array, wherein each of the plurality of needles includes a needle tip at one end, and tapers at a taper angle and along a taper length to a maximum needle diameter at the other end, and wherein the taper angle ranges from approximately 5 degrees to approximately 15 degrees.

In another aspect, a needling device includes a plurality of needles forming a needle array, and a motor assembly for driving the needle array, wherein each of the plurality of needles includes a needle tip at one end, and tapers at a taper angle and along a taper length to a maximum needle diameter at the other end, and wherein the needle tip has a tip radius ranging from approximately 0.015 mm to approximately 0.025 mm.

In another aspect, a needling device includes a plurality of needles forming a needle array, and a motor assembly for driving the needle array, wherein, in use, a skin reference surface of the needling device is in contact with a subject's skin, and the skin reference surface has a surface area ranging from approximately 45 mm² to approximately 105 mm².

In another aspect, a needling device includes a plurality of needles forming a needle array, and a motor assembly for driving the needle array, wherein, in use, a skin reference surface of the needling device is in contact with a subject's skin, and an average distance between each needle of the plurality of needles of the needle array and the skin reference surface area ranges from approximately 0.10 mm to approximately 2.5 mm.

In another aspect, a needling device includes a plurality of needles forming a needle array, and a motor assembly for driving the needle array, wherein the motor assembly includes a motor linkage, and wherein the motor linkage includes a rotational component with a total mass ranging from approximately 0.5 grams to approximately 35 grams and a rotational radius ranging from approximately 1.5 mm to approximately 3.5 mm, and the motor linkage includes a linear component with a total mass ranging from approximately 1.5 grams to approximately 3.5 grams.

### 4. BRIEF DESCRIPTION OF THE SEVERAL VIEWS OF THE DRAWINGS

The foregoing summary, as well as the following detailed description, will be better understood when read in conjunction with the appended drawings. For the purpose of illustration, there is shown in the drawings certain embodiments of the present disclosure. It should be understood, however, that the invention is not limited to the precise arrangements and instrumentalities shown. The accompanying drawings, which are incorporated in and constitute a part of this specification, illustrate an implementation of systems and apparatuses consistent with the present invention and, together with the description, serve to explain advantages and principles consistent with the invention.

FIG. 1A illustrates a schematic view of a subject's skin, the epidermis, dermis, bulge, and sebaceous gland.

FIG. 1B is a diagram illustrating an example of skin puncture dynamics.

FIG. 2A is a diagram illustrating a perspective view of a manufacturer's needle.

FIG. 2B is a diagram illustrating a perspective view of an example of a needle according to the present disclosure.

FIG. 3 is a diagram illustrating a schematic view of the manufacturer's needle of FIG. 2A.

FIG. 4 is a diagram illustrating a schematic view of another manufacturer's needle.

FIG. 5 is a diagram illustrating a schematic view of the example of the needle of FIG. 2B.

FIG. 6 is a diagram illustrating a schematic view of another example of a needle according to the present disclosure.

FIG. 7 is a diagram illustrating a schematic view of another example of a needle according to the present disclosure.

FIG. 8 is a diagram illustrating a schematic view of another example of a needle according to the present disclosure.

FIG. 9 is a diagram illustrating a schematic view of another example of a needle according to the present disclosure.

FIG. 10 is a diagram illustrating a schematic view of another example of a needle according to the present disclosure.

FIG. 11 is a diagram illustrating a schematic view of another example of a needle according to the present disclosure.

FIG. 12 is a diagram illustrating a schematic view of another example of a needle according to the present disclosure.

FIG. 13 is a diagram illustrating a schematic view of another example of a needle according to the present disclosure.

FIG. 14 is a diagram illustrating a schematic view of another example of a needle according to the present disclosure.

FIG. 15 is a diagram illustrating a schematic view of another example of a needle according to the present disclosure.

FIG. 16 is a diagram illustrating a schematic view of another example of a needle according to the present disclosure.

FIG. 17 is a diagram illustrating a schematic view of another example of a needle according to the present disclosure.

FIG. 18 is a diagram illustrating a schematic view of another example of a needle according to the present disclosure.

FIG. 19A is a diagram illustrating a skin reference surface of a manufacturer A's needling device.

FIGS. 19B, 19C, and 19D are diagrams illustrating a skin reference surface of a needling device according to an example the present disclosure.

FIG. 20 is a diagram illustrating an example of average needle distance from the reference surface according to an example of the present disclosure

FIGS. 21A, 21B, 21C, 21D, 21E, 21F, and 21G are diagrams illustrating an example of a needle depth measurement tissue study.

FIG. 22 is an annotated example of a single tissue section used for a needle depth measurement tissue study.

FIG. 23 shows histograms of the histologically measured dye penetration for the three different depth setting of the core.

FIG. 24 illustrates that a needling device according to an example of the present disclosure functions as intended by the user depth setting.

FIG. 25 is a diagram illustrating the results of an exemplary comparative needle depth study.

FIG. 26 is a diagram illustrating a motor linkage of a needling device according to an example of the present disclosure, and a motor linkage of other manufacturer's needling devices.

FIG. 27 is a graph illustrating the rotational inertia and linear mass of the motor linkages of FIG. 26.

FIG. 28 is a graph illustrating the tolerance collapse of the motor linkages of FIG. 26.

FIG. 29A is a diagram illustrating a schematic view of another example of a motor linkage of a needling device according to the present disclosure.

FIG. 29B is a diagram illustrating a schematic view of a further example of a motor linkage of a needling device according to the present disclosure.

### 5. DETAILED DESCRIPTION OF THE INVENTION

Before explaining at least one embodiment of the invention in detail, it is to be understood that the invention is not limited in its application to the details of construction and to the arrangements of the components set forth in the following description or illustrated in the drawings. The Figures and written description are provided to teach any person skilled in the art to make and use the inventions for which patent protection is sought. The invention is capable of other embodiments and of being practiced and carried out in various ways. Those skilled in the art will appreciate that not all features of a commercial embodiment are shown for the sake of clarity and understanding.

In addition, it is to be understood that the phraseology and terminology employed herein are for the purpose of description and should not be regarded as limiting. For example, the use of a singular term, such as, “a” is not intended as limiting of the number of items. Also the use of relational terms, such as but not limited to, “top,” “bottom,” “left,” “right,” “upper,” “lower,” “down,” “up,” “side,” are used in the description for clarity in specific reference to the Figures and are not intended to limit the scope of the invention or the appended claims. The terms “approximately,” “substantially,” “about”, and “around,” set a value described as such to equal any value ranging from plus or minus 5%. For example, a value of about 10 mm is equal to any value from 9.5 mm to 10.5 mm. Further, it should be understood that any one of the features of the invention may be used separately or in combination with other features. Other systems, methods, features, and advantages of the invention will be or become apparent to one with skill in the art upon examination of the Figures and the detailed description. It is intended that all such additional systems, methods, features, and advantages be included within this description, be within the scope of the present invention, and be protected by the accompanying claims.

### 5.1 Overview

Needling devices described herein may be used for a number of different procedures including, for example, hair growth applications, wrinkle reduction, scar revision, hair removal, tattoo removal, and pigmentation. Advantages of the needling devices described in this application include providing a set of needles and needle configuration, and a motor configuration that allows optimal and precise needling and achieving therapeutic depth and puncture precision in treatment.

It was believed until quite recently that follicle formation occurs but once in a lifetime (in utero), so that a mammal, and particularly a human, is born with a fixed number of follicles, which does not normally increase thereafter. Despite suggestions of the regenerative capacity of the adult mammalian skin to recreate the embryonic follicle, until recently, follicle neogenesis was not proven because of the lack of tools needed to demonstrate the occurrence or hair follicle neogenesis (see, Argyris et al, 1959, Dev. Biol. 1: 269-80; Miller, 1973, J. Invest. Dermatol. 58: 1-9; and ligman, 1959, Ann NY Acad Sci 83: 507-511).

It has been proposed, however, that hair follicle neogenesis can be associated with wound healing in animals (e.g., rabbits, mice). See, Stenn & Paus, 2001, Physiol. Revs. 81:449-494. More recently, a series of murine experiments definitively showed that hair follicle-derived epithelial stem cell progenitors migrate out of the follicle and contribute to the re-epithelialization of injured skin (see, Morris et al, 2004, Nature Biotechnology 22:411-417; Ito et al, 2004, Differentiation 72:548-57; and Ito et al, 2005, Nature Medicine 11: 1351-1354). In animal studies designed to explore the role of Wnt in hair follicle development, Fathke showed that prolonged activation of Wnt signaling during wound healing in mice resulted in generation of rudiments of hair follicles but did not result in the formation of hair follicles or growth of more hair (Fathke et al, 2006, BMC Cell Biol. 7:4).

As noted by Fathke, cutaneous repair in adult mammals following full thickness wounding is understood to result in scar tissue and the loss of the regenerative capability of the hair follicle. Severe wounds and burns are usually associated with a form of cutaneous repair that results in scar tissue and no hair follicles (see, Fathke et al, 2006, BMC Cell Biol. 7:4). However, in a mouse study, Cotsarelis showed that physically disrupting the skin and existing follicles, in a defined fashion, can lead to follicle neogenesis (Ito et al, 2007, Nature 447:316-321). Cotsarelis showed that following closure of large healed wounds created by full thickness excision (FTE) (1 cm2 square wounds) in mice, new hairs are formed at the center of the wound (Ito et al, 2007, Nature 447:316-321). (Argyris, 1976, Amer J Pathol 83:329-338).

Other preclinical studies have identified a therapeutic window after epithelial disruption where the skin reverts to an embryonic state, allowing manipulation of skin and follicle phenotype by addition of compounds. For example, because new hair patterns after wounding are not predetermined, the regulatory pathways relevant to follicle formation (e.g. Wnt, EGFR) can be influenced dramatically, e.g., to increase the number and size of follicles. See, Ito et al. Nature. 2007; 447(7142):316-320; Fathke et al. BMC Cell Biol. 2006; 7:4; Snippert et al. Science. 2010; 327(5971): 1385-1389.

The needling devices, the needles, and the methods described herein provide optimal and precise needling to achieve optimized therapeutic outcome.

### 5.1.1 Use of Needling Devices

Needling devices in accordance with various examples of the present disclosure may be used for hair growth, wrinkle reduction, scar revision, hair removal, tattoo removal, among other treatments.

Needling devices and treatments using needling devices in accordance with various examples of the present disclosure may also be used in combination with one or more agents. In one aspect, the agent is an agent that promotes hair growth. In one aspect, the agent is an agent that is useful in reducing wrinkles. In one aspect, the agent is an agent that is useful in scar revision. In one aspect, the agent is an agent that is useful in hair removal. In one aspect, the agent is an agent that is useful in tattoo removal. In one aspect, the agent is an agent that is useful in pigmentation. In another aspect, the agent is a topical anesthetic.

### 5.1.2 Therapeutic Depth

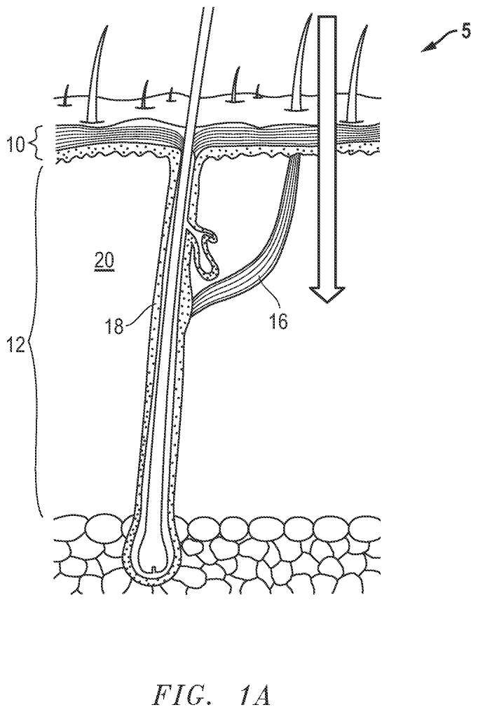

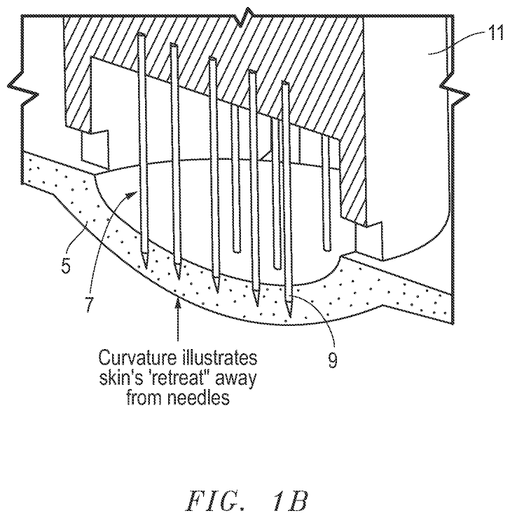

FIG. 1A illustrates a schematic view of a subject's skin 5, the epidermis 10, dermis 12, bulge 20, and sebaceous gland 18. Referring to FIG. 1A, the epidermis 10 is at a depth of up to approximately 0.05 mm, the dermis 12 is at a depth of approximately 1.3 mm to 1.5 mm, the bulge 20 is at a depth of approximately 0.6 mm to 0.8 mm, and the sebaceous gland 18 is at a depth of approximately 0.06 mm. Also illustrated in FIG. 1A is the arrector pili muscle 16.

Stem cells are thought to be activated in the hair bulge 20 under wound healing conditions, along with the induction of hair growth-related genes, such as VEGF, beta-catenin, and Wnt signaling molecules. The most important stem cells are located at the bulge 20 so that it is desirable to disrupt the skin 5 deep enough to disrupt the sebaceous gland 18, bulge 20, or hair papilla of existing follicle structures.

At the same time, it is important to minimize adverse effects. An optimization of clinical effects while minimizing adverse effects is desirable. It is also desirable to localize wound healing effects at the most therapeutically relevant depth and optimize the clinical effect while minimizing the potential for adverse effects that result therefrom.

The needling devices, needles, and methods described herein provide optimal puncture depth so as to maximize therapeutic effect while minimizing adverse effects

### 5.1.3 Skin Puncture Dynamics

FIG. 1B is a diagram illustrating an example of skin puncture dynamics. The initial dynamics at first encounter between the surface of the skin 5 and the tip of a needle 9 is important. This includes disruption of the stratum corneum, the thin outer protective layer of the skin at approximately the first 10-30 μm of skin cells. The skin 5 overall is an elastic material and in particular the top stratum corneum layer resists puncture, allowing deformation away from an attempted needle 9 puncture. Softer layers of subcutaneous tissue under the skin 5 may further enable deformation away from the attempted puncture of a needle 9.

Referring to FIG. 1B, in the case of rapidly oscillating microneedles 9 from a powered microneedling device 11, if needles 9 are not able to penetrate the protective layer of the skin 5 upon initial impact, elastic deformation away from the downward travel of needles 9 is accentuated. This increases the degree to which the skin is able to retreat away from the needles, creates a curving away region 7 resulting from the skin 5 retreating away, and reduces the needles' 9 actual puncture depth.

Accordingly, even if different treatments are intended to achieve the same disruption depth, clinical effects and actual results will vary depending on the initial dynamic at first encounter between the skin surface and needle tip. Initial dynamics are affected by aspects of a microneedling device including, but not limited to, needle dimensions as described in Section 5.2.1, needle array orientation as described in Section 5.2.2, and the motor and motor linkage as described in Section 5.2.3. In addition to the significance of treatment depth as measured by needle extension tests, the actual penetration of the stratum corneum provides improved clinical effects depending on initial skin puncture dynamics.

### 5.2 Needling Device

### 5.2.1 Needle Dimensions

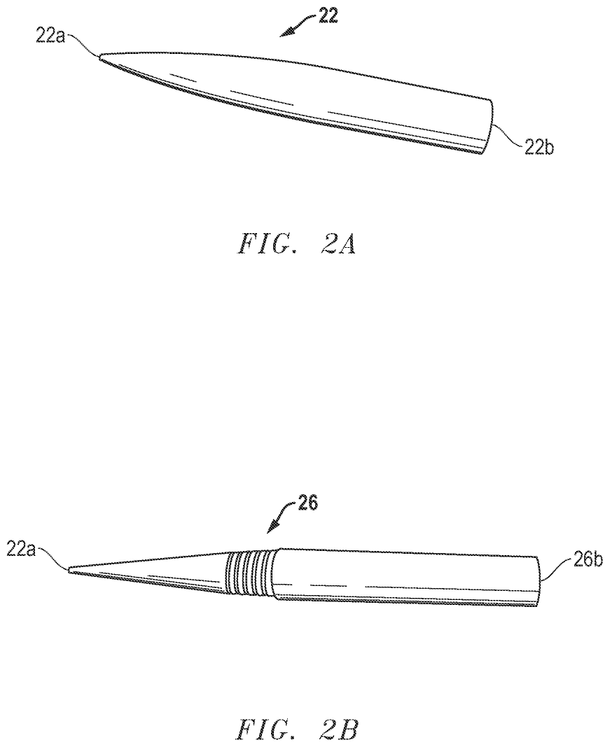

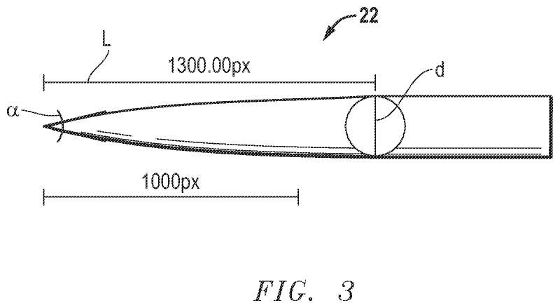

FIG. 2A is a diagram illustrating a perspective view of a manufacturer A's needle 22. Referring to FIG. 2A, a manufacturer's needle 22, as used in a micro-needling device, has a shape with a sharp, narrow tip at the skin entry end 22a which tapers to a wider needle diameter at the opposite end 22b. The dimensions of the manufacturer needle 22 will be described in more detail below in reference with FIG. 3.

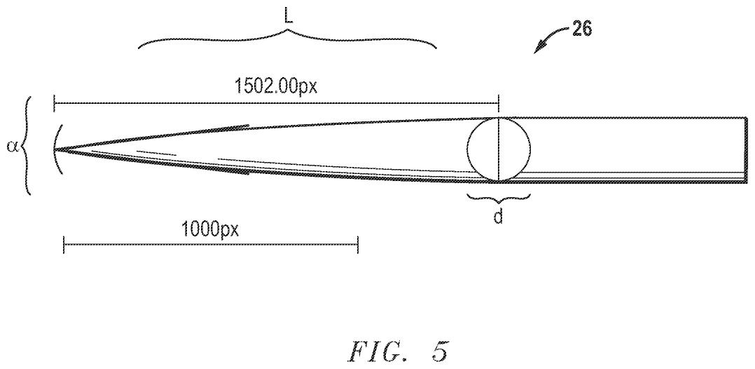

FIG. 2B is a diagram illustrating a perspective view of an example of a needle 26 according to the present disclosure. Referring to FIG. 2B, compared to the manufacturer needle 22, the needle 26 has a more blunt needle tip at the skin entry end 26a, a smaller taper angle, and a smaller diameter at the opposite end 26b. In some examples, the taper length of the needle 26 according to an example the present disclosure is longer than the taper length of the manufacturer needle 22. A number of examples of needles according to the present disclosure are described in more detail below in reference with FIGS. 5-18.

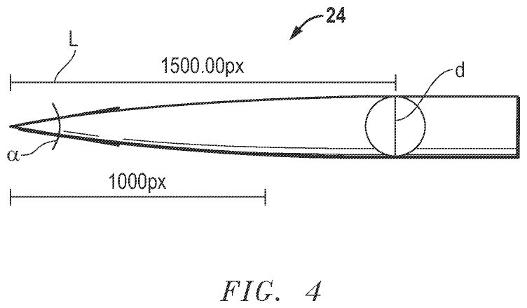

FIG. 3 is a diagram illustrating a schematic view of the manufacturer A's needle 22 of FIG. 2A. FIG. 4 is a diagram illustrating a schematic view of a manufacturer B's needle 24. Referring to FIG. 3, the manufacturer's needle 22 has a taper angle α of approximately 23.84 degrees, a taper length L of approximately 1.298 mm, and a diameter d of approximately 0.249 mm. Referring to FIG. 4, the manufacturer's needle 24 has a taper angle α of approximately 16.61 degrees, a taper length L of approximately 1.491 mm, and a diameter d of approximately 0.250 mm. An outline of the dimensions of manufacturer A's needle 22 and manufacturer B's needle 24 is provided, as follows:

TABLE 1
Manufacturer A and Manufacturer B Needle Dimensions

| | Diameter d | Taper length L | Point Angle α |
|---|---|---|---|
| | (mm) | (mm) | (degrees) |
| Manufacturer A | 0.249 | 1.298 | 23.84 |
| Needle (22) | | | |
| Manufacturer B | 0.250 | 1.491 | 16.61 |
| Needle (24) | | | |

FIG. 5 is a diagram illustrating a schematic view of the needle 26 of FIG. 2B according to an example of the present disclosure. Referring to FIG. 5, the needle 26 has a taper angle α of approximately 9.78 degrees, a taper length L of approximately 1.390 mm, and a diameter d of approximately 0.223 mm. An outline of the dimensions of the needle diameter for manufacturer A's needle 22, manufacturer B's needle 24, and needle 26 is provided, as follows:

TABLE 2
Needle Diameter for Manufacturer A, Manufacturer B, And Exemplary Needle
Needle Diameter d (mm)

| | ASTM/American | Birmingham | Manufacturer A | Manufacturer B | |
|---|---|---|---|---|---|
| Gage | Standard | Standard | Needle (22) | Needle (24) | Needle (26) |
| 31 | 0.226 | 0.2604 | | | |
| 32 | 0.203 | 0.235 | 0.249 | 0.25 | 0.223 |
| 33 | 0.18 | 0.2096 | | | |

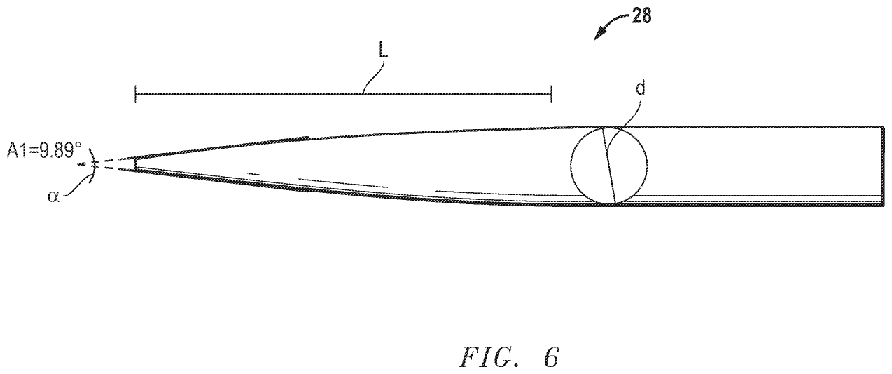

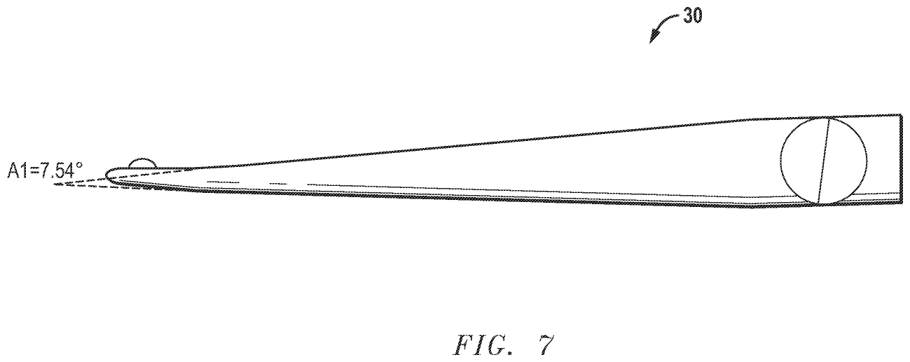

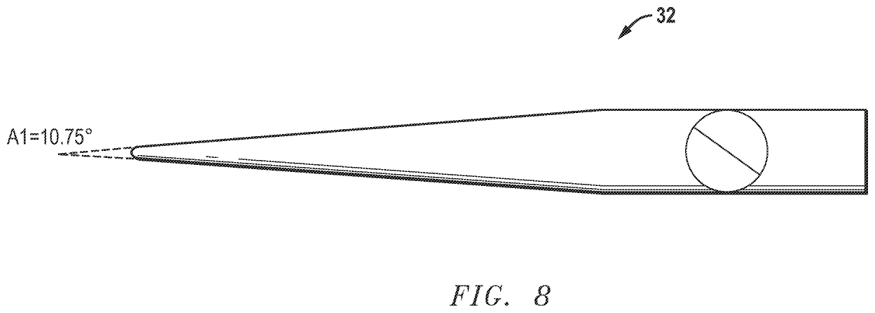

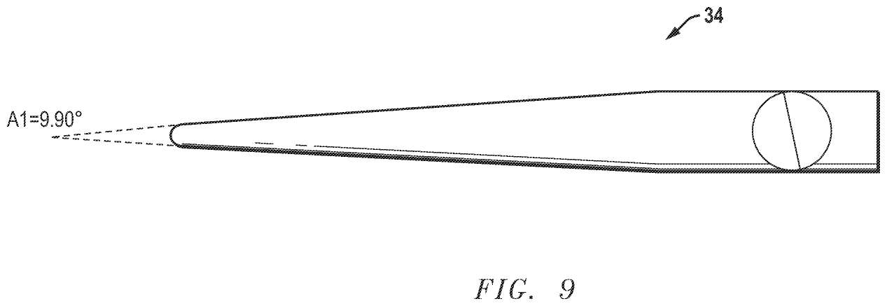

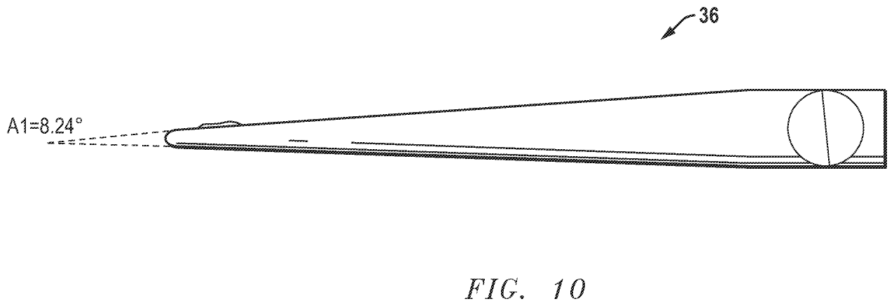

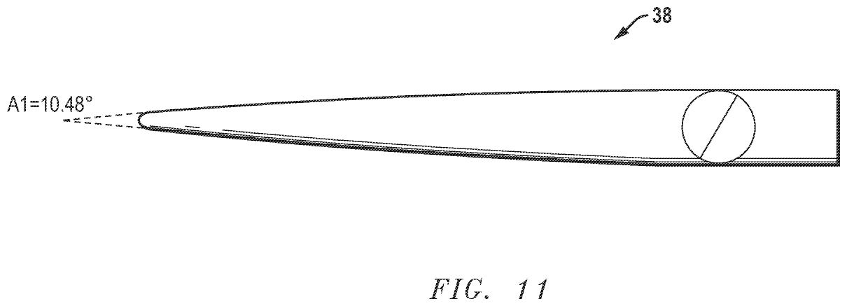

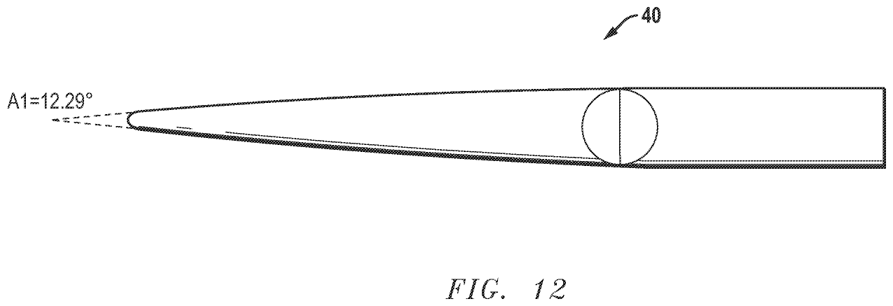

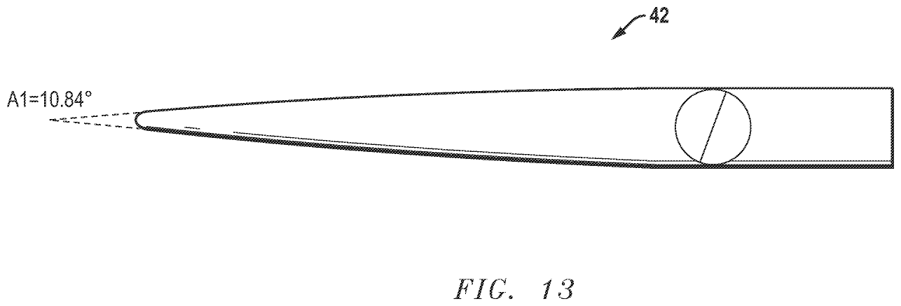

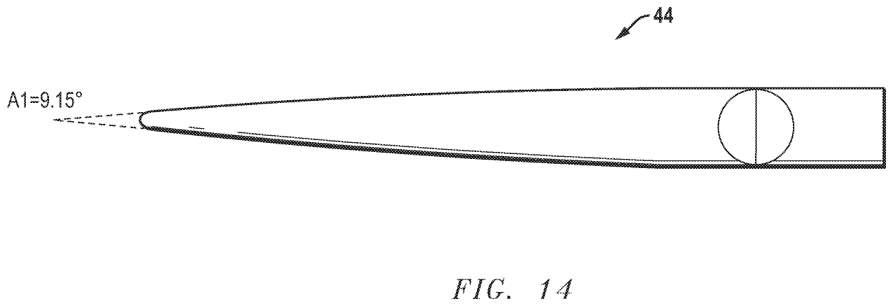

FIGS. 6-18 are diagrams illustrating schematic views of other examples of needles 28, 30, 32, 34, 36, 38, 40, 42, 44, 46, 48, 50, 54 according to the present disclosure. Referring to FIG. 6, the needle 28 has a taper angle α of approximately 9.89 degrees, a taper length L of approximately 1.265 mm, and a diameter d of approximately 0.214 mm. Referring to FIG. 7, the needle 30 has a taper angle α of approximately 7.54 degrees, a taper length L of approximately 1.665 mm, and a diameter d of approximately 0.226 mm. Referring to FIG. 8, the needle 32 has a taper angle α of approximately 10.75 degrees, a taper length L of approximately 1.308 mm, and a diameter d of approximately 0.225 mm. Referring to FIG. 9, the needle 34 has a taper angle α of approximately 9.90 degrees, a taper length L of approximately 1.389 mm, and a diameter d of approximately 0.221 mm. Referring to FIG. 10, the needle 36 has a taper angle α of approximately 8.24 degrees, a taper length L of approximately 1.750 mm, and a diameter d of approximately 0.221 mm. Referring to FIG. 11, the needle 38 has a taper angle α of approximately 10.48 degrees, a taper length L of approximately 1.417 mm, and a diameter d of approximately 0.220 mm. Referring to FIG. 12, the needle 40 has a taper angle α of approximately 12.29 degrees, a taper length L of approximately 1.210 mm, and a diameter d of approximately 0.228 mm. Referring to FIG. 13, the needle 42 has a taper angle α of approximately 10.84 degrees, a taper length L of approximately 1.430 mm, and a diameter d of approximately 0.223 mm. Referring to FIG. 14, the needle 44 has a taper angle α of approximately 9.15 degrees, a taper length L of approximately 1.390 mm, and a diameter d of approximately 0.222 mm. Referring to FIG. 15, the needle 46 has a taper angle α of approximately 10.26 degrees, a taper length L of approximately 1.042 mm, and a diameter d of approximately 0.224 mm. Referring to FIG. 16, the needle 48 has a taper angle α of approximately 8.25 degrees, a taper length L of approximately 1.614 mm, and a diameter d of approximately 0.224 mm. Referring to FIG. 17, the needle 50 has a taper angle α of approximately 10.05 degrees, a taper length L of approximately 1.212 mm, and a diameter d of approximately 0.226 mm.

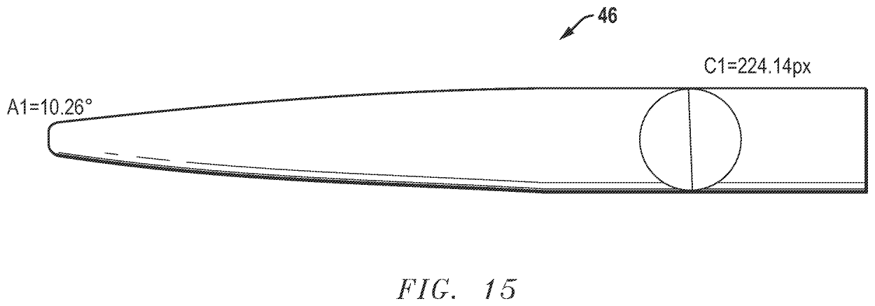

Referring to FIG. 18, the needle 52 may have an overall needle length of approximately 7.43 mm, a taper angle α of approximately 9.50 degrees, a taper length L of approximately 1.100 mm, and a diameter d of approximately 0.223 mm. Further, the needle tip radius r may be approximately 0.02 mm so that the needle tip is a relatively blunt or slightly rounded tip. According to one example, a slightly rounded tip, at the point of greatest velocity and kinetic energy, will have a greater impact on the stratum corneum barrier. An outline of the dimensions of the needles 28, 30, 32, 34, 36, 38, 40, 42, 44, 46, 48, 50, 54 is provided, as follows:

TABLE 3
Exemplary Needle Dimensions

| | Diameter d | Taper length L | Point Angle α |
|---|---|---|---|
| | (mm) | (mm) | (Degrees) |
| Needle (26) | 0.223 | 1.390 | 9.78 |
| Needle (28) | 0.214 | 1.265 | 9.89 |
| Needle (30) | 0.226 | 1.665 | 7.54 |
| Needle (32) | 0.225 | 1.308 | 10.75 |
| Needle (34) | 0.221 | 1.389 | 9.90 |
| Needle (36) | 0.221 | 1.750 | 8.24 |
| Needle (38) | 0.220 | 1.417 | 10.48 |
| Needle (40) | 0.228 | 1.210 | 12.29 |
| Needle (42) | 0.223 | 1.430 | 10.84 |
| Needle (44) | 0.222 | 1.390 | 9.15 |
| Needle (46) | 0.224 | 1.042 | 10.26 |
| Needle (48) | 0.224 | 1.614 | 8.25 |
| Needle (50) | 0.226 | 1.212 | 10.05 |
| Needle (52) | 0.223 | 1.100 | 9.50 |

In the above described examples, a longer taper length L and a smaller taper angle α may enable a needle to less abruptly puncture the dermis over the course of needle penetration. A smaller diameter d may optimize the diameter for greater skin disruption with the ability to achieve full puncture. A slightly rounded initial tip may create more significant initial puncture to allow the needle to puncture more deeply. According to various examples, the needle diameter d may range from approximately 0.20 mm to approximately 0.24 mm. The diameter d includes at least 0.20 mm, at least 0.21 mm, at least 0.22 mm, at least 0.23 mm, at least 0.24 mm, at most 0.20 mm, at most 0.21 mm, at most 0.22 mm, at most 0.23 mm, and at most 0.24 mm. According to various examples, the taper length L may range from approximately 1 mm to approximately 2 mm. The taper length L includes at least 1 mm, at least 1.1 mm, at least 1.2 mm, at least 1.3 mm, at least 1.4 mm, at least 1.5 mm, at least 1.6 mm, at least 1.7 mm, at least 1.8 mm, at least 1.9 mm, at least 2 mm, at most 1 mm, at most 1.1 mm, at most 1.2 mm, at most 1.3 mm, and at most 1.4 mm, at most 1.5 mm, at most 1.6 mm, at most 1.7 mm, at most 1.8 mm, at most 1.9 mm, and at most 2 mm. According to various examples, the taper angle α may range from approximately 5 degrees to approximately 15 degrees. The taper angle α includes at least 5 degrees, at least 6 degrees, at least 7 degrees, at least 8 degrees, at least 9 degrees, at least 10 degrees, at least 11 degrees, at least 12 degrees, at least 13 degrees, at least 14 degrees, at least 15 degrees, at most 5 degrees, at most 6 degrees, at most 7 degrees, at most 8 degrees, at most 9 degrees, at most 10 degrees, at most 11 degrees, at most 12 degrees, at most 13 degrees, at most 14 degrees, at most 15 degrees. According to various examples, the needle tip radius r may range from approximately 0.015 mm to 0.025 mm. The needle tip radius r includes at least 0.015 mm, at least 0.016 mm, at least 0.017 mm, at least 0.018 mm, at least 0.019 mm, at least 0.02 mm, at least 0.021 mm, at least 0.022 mm, at least 0.023 mm, at least 0.024 mm, at least 0.025 mm, at most 0.015 mm, at most 0.016 mm, at most 0.017 mm, at most 0.018 mm, at most 0.019 mm, at most 0.02 mm, at most 0.021 mm, at most 0.022 mm, at most 0.023 mm, at most 0.024 mm, at most 0.025 mm.

### 5.2.2 Needle Array Orientation

A microneedling device is used by translating the device across a subject's skin in gliding strokes, maintaining contact with the skin. The part of the device in contact with the skin may be referred to as the skin reference surface, through which the vertically oscillating needles extend. For example, at some points in the oscillation cycle, the skin reference surface may be the only part of the device, other than the needles themselves, that is in contact with a subject's skin. One function of the skin reference surface is to control the exposed extension of the needles and to prevent excessive, unintended depth of penetration. Given the skin's tendency to retreat upon puncture, an optimized skin reference surface must also effectively hold the skin in place, preventing a greater amount of skin from stretching and allowing further “retreat”. This skin dynamic in response to initial puncture of the skin is also described above in Section 5.1.3 and in reference with FIG. 1B.

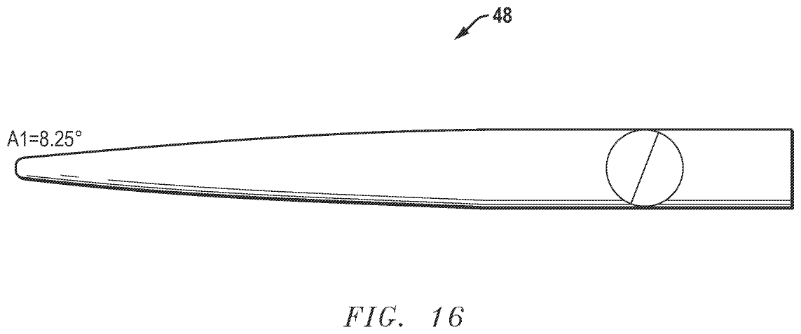

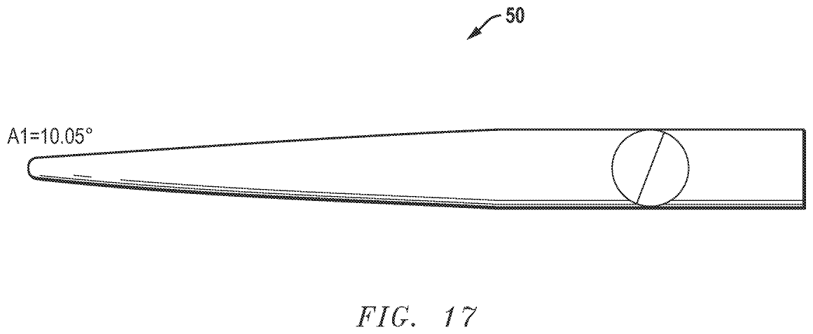

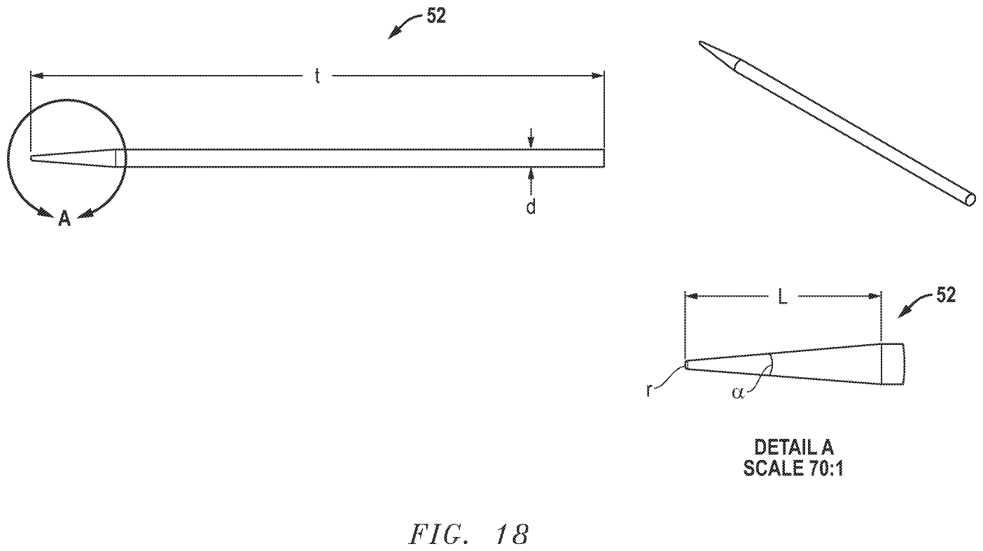

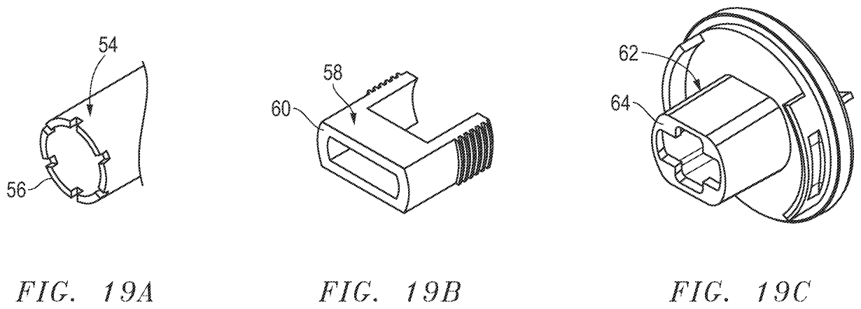

FIG. 19A is a diagram illustrating a skin reference surface 56 of a manufacturer A's needling device 54. As illustrated in FIG. 19A, the skin reference surface 56 in manufacturer A's needling device 54 has a small surface area. Accordingly, the part of the device in contact with and holding the skin is small. The surface area of the skin reference surface 56 is 27 mm². As the reference surface must be able to glide across the skin, its ability to hold the skin momentarily upon puncture is based on instantaneous adhesion forces between the skin and the skin reference surface, which increase with a greater cross-sectional area of the reference surface. As a result, needle depth and skin surface dynamics are adversely affected when using a needling device having a skin reference surface 56 with a small surface area such as manufacturer A's needling device 54.

FIGS. 19B, 19C, and 19D are diagrams illustrating a skin reference surface 60, 64, 68 of a needling device 58, 62, 64 according to an example of the present disclosure. Referring to FIGS. 19B-19D, the skin reference surface 60, 64, 68 in a needling device 58, 62, 64 according to an example of the present disclosure has a large surface area. Accordingly, the part of the device in contact with and holding the skin is large. In an example, the surface area of the skin reference surface 58, 62 is approximately 75 mm². In an example, the surface area of the skin reference surface 64 is approximately 230 mm².

According to various examples, the surface area of the skin reference surface 58, 62 may range from approximately 45 mm² to approximately 105 mm². The surface area includes at least 45 mm², at least 50 mm², at least 55 mm², at least 60 mm², at least 65 mm², at least 70 mm², at least 75 mm², at least 80 mm², at least 85 mm², at least 90 mm², at least 95 mm², at least 100 mm², at least 105 mm², at least 110 mm², at least 115 mm², at least 120 mm², at least 125 mm², at least 130 mm², at least 135 mm², at least 140 mm², at least 145 mm², at least 150 mm², at least 155 mm², at least 160 mm², at least 165 mm², at least 170 mm², at least 175 mm², at least 180 mm², at least 185 mm², at least 190 mm², at least 195 mm², at least 200 mm², at least 205 mm², at least 210 mm², at least 215 mm², at least 220 mm², at least 225 mm², at least 230 mm², at least 235 mm², at least 240 mm², at least 245 mm², at least 250 mm², at least 255 mm², at least 260 mm², at least 265 mm², at least 270 mm², at least 275 mm², at most 45 mm², at most 50 mm², at most 55 mm², at most 60 mm², at most 65 mm², at most 70 mm², at most 75 mm², at most 80 mm², at most 85 mm², at most 90 mm², at most 95 mm², at most 100 mm², at most 105 mm², at most 110 mm², at most 115 mm², at most 120 mm², at most 125 mm², at most 130 mm², at most 135 mm², at most 140 mm², at most 145 mm², at most 150 mm², at most 155 mm², and at most 160 mm² at most 165 mm², at most 170 mm², at most 175 mm², at most 180 mm², at most 185 mm², at most 190 mm², at most 195 mm², at most 200 mm², at most 205 mm², at most 210 mm², at most 215 mm², at most 220 mm², at most 225 mm², at most 230 mm², at most 235 mm², at most 240 mm², at most 245 mm², at most 250 mm², at most 255 mm², at most 260 mm², at most 265 mm², at most 270 mm², at most 275 mm². As a result, the needling device 58, 62, 66 is optimized to have a greater skin reference surface area, thereby holding onto the skin and reducing its ability to retreat at the instant of puncture.

Another factor affecting the degree to which the skin can “retreat” is the effective distance between the reference surface and the needles. A needle that is close to the reference surface has a shorter length of skin. Governed by its modulus of elasticity, a shorter length of skin is less able to “tent” away from the penetrating needle than a longer distance, and therefore is more likely to receive greater needle puncture.

FIG. 20 is a diagram illustrating an example of average needle distance from the reference surface according to an example of the present disclosure. Referring to FIG. 20, the reference surface 74 surrounds the needles 70, 72 and is the part of the device in contact with the skin. In an example, the average needle to reference surface distance may be calculated by adding each of the distances that each needle 70, 72 is spaced away from the reference surface 74 and dividing by the number of needles 70, 72. The needle 70, 72 distance from the reference surface 74 is measured by measuring the shortest distance between the needle 70, 72 and any part of the reference surface 74. Still referring to FIG. 20, the needle to reference surface distance for needle 70 is illustrated by arrow x, and the needle to reference surface distance for needle 72 is illustrated by arrow y.

Referring back to FIGS. 19B, 19C, and 19D, the average needle to reference surface distance for the needling device 58 is approximately 1.12 mm, the average needle to reference surface distance for the needling device 62 is approximately 2.23 mm, and the average needle to reference surface distance for the needling device 66 is approximately 0.20 mm.

According to various examples, the average needle to reference surface distance may range from approximately 0.10 mm to 2.5 mm. The average needle to reference surface distance includes at least 0.1 mm, at least 0.2 mm, at least 0.3 mm, at least 0.4 mm, at least 0.5 mm, at least 0.6 mm, at least 0.7 mm, at least 0.8 mm, at least 0.9 mm, at least 1.0 mm, at least 1.1 mm, at least 1.2 mm, at least 1.3 mm, at least 1.4 mm, at least 1.5 mm, at least 1.6 mm, at least 1.7 mm, at least 1.8 mm, at least 1.9 mm, at least 2.0 mm, at least 2.1 mm, at least 2.2 mm, at least 2.3 mm, at least 2.4 mm, at least 2.5 mm, at most 0.1 mm, at most 0.2 mm, at most 0.3 mm, at most 0.4 mm, at most 0.5 mm, at most 0.6 mm, at most 0.7 mm, at most 0.8 mm, at most 0.9 mm, at most 1.0 mm, at most 1.1 mm, at most 1.2 mm, at most 1.3 mm, at most 1.4 mm, at most 1.5 mm, at most 1.6 mm, at most 1.7 mm, at most 1.8 mm, at most 1.9 mm, at most 2.0 mm, at most 2.1 mm, at most 2.2 mm, at most 2.3 mm, at most 2.4 mm, at most 2.5 mm. By using a linear array of needles with a close-fitting reference surface, the needling device 58, 62, 66 has a lower, optimized distance between its needles and the closest edge of the reference surface compared with other devices such as devices having a circular orientation of needles.

### 5.2.3 Motor and Linkage

In an example, a needling device according to the present disclosure may have a motor and a motor linkage which provides a tighter attachment and linkage between all components of the motor assembly. A greater inertia of linkage, motor, and moving shaft increases the kinetic energy at time of impact and increases the ability to effect full puncture. In addition, a greater stiffness of linkage reduces the mechanical yield of the linkage upon impact and increases the ability to transfer kinetic energy to the skin and disrupt the surface, thus reducing skin's ability to retreat and allowing greater penetration.

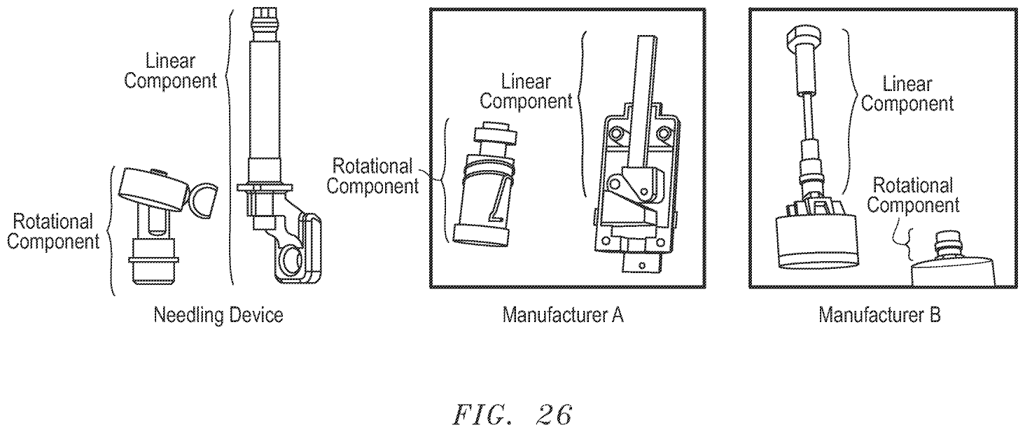

FIG. 26 is a diagram illustrating a motor linkage of a needling device according to an example of the present disclosure, and a motor linkage of other manufacturer's needling devices. Referring to FIG. 26, a needling device according to an example of the present disclosure includes a rotational component and a linear component. Similarly, each of the other manufacturer's devices also include a rotational component and a linear component.

Still referring to FIG. 26, an increase in the linkage inertia aids in depth realization assuming other conditions are the same; for example, the motor, the software, and the needles. Inertia may correlate generally with how likely the motor linkage is to push the needles through the skin's protective layer. Rotational inertia is calculated as I=½ mr² where I is inertia, m is mass, and r is radius of the rotating object, and linear inertia is directly related with the mass of the object moving linearly. Accordingly, with a greater mass and radius of the rotational component, and a greater mass of the linear component, a greater inertial is achieved.

According to various examples, the mass of the rotational component in a motor linkage of a needling device according to an example of the present disclosure may range from approximately 0.5 grams to approximately 35 grams. The mass of the rotational component includes at least 0.5 g, at least 1.0 g, at least 2.0 g, at least 3.0 g, at least 4.0 g, at least 5.0 g, at least 6.0 g, at least 7.0 g, at least 8.0 g, at least 9.0 g, at least 10.0 g, at least 11.0 g, at least 12.0 g, at least 13.0 g, at least 14.0 g, at least 15.0 g, at least 16.0 g, at least 17.0 g, at least 18.0 g, at least 19.0 g, at least 20.0 g, at least 21.0 g, at least 22.0 g, at least 23.0 g, at least 24.0 g, at least 25.0 g, at least 26.0 g, at least 27.0 g, at least 28.0 g, at least 29.0 g, at least 30.0 g, at least 31.0 g, at least 32.0 g, at least 33.0 g, at least 34.0 g, at least 35.0 g, at most 0.5 g, at most 1.0 g, at most 2.0 g, at most 3.0 g, at most 4.0 g, at most 5.0 g, at most 6.0 g, at most 7.0 g, at most 8.0 g, at most 9.0 g, at most 10.0 g, at most 11.0 g, at most 12.0 g, at most 13.0 g, at most 14.0 g, at most 15.0 g, at most 16.0 g, at most 17.0 g, at most 18.0 g, at most 19.0 g, at most 20.0 g, at most 21.0 g, at most 22.0 g, at most 23.0 g, at most 24.0 g, at most 25.0 g, at most 26.0 g, at most 27.0 g, at most 28.0 g, at most 29.0 g, at most 30.0 g, at most 31.0 g, at most 32.0 g, at most 33.0 g, at most 34.0 g, and at most 35.0 g. According to various examples, the radius of the rotational component in a motor linkage of a needling device according to an example of the present disclosure may range from approximately 1.5 mm to approximately 3.5 mm. The radius of the rotational component includes at least 1.5 mm, at least 2.0 mm, at least 2.5 mm, at least 3.0 mm, at least 3.5 mm, at most 1.5 mm, at most 2.0 mm, at most 2.5 mm, at most 3.0 mm, at most 3.5 mm. Based on the rotational mass and rotational radius used, a rotational inertia may be calculated using the inertia formula provided above. In one example where the rotational mass is approximately 1.34 g and the rotational radius is approximately 2.5 mm, the rotational inertia is approximately 4.19E-06 g*m². According to various examples, the mass of the linear component in a motor linkage of a needling device according to an example of the present disclosure may range from approximately 1.5 grams to approximately 3.5 grams. The mass of the linear component includes at least 1.5 g, at least 2.0 g, at least 2.5 g, at least 3.0 g, at least 3.5 g, at most 1.5 g, at most 2.0 g, at most 2.5 g, at most 3.0 g, at most 3.5 g. In an example, the linear mass may be approximately 2.25 g.

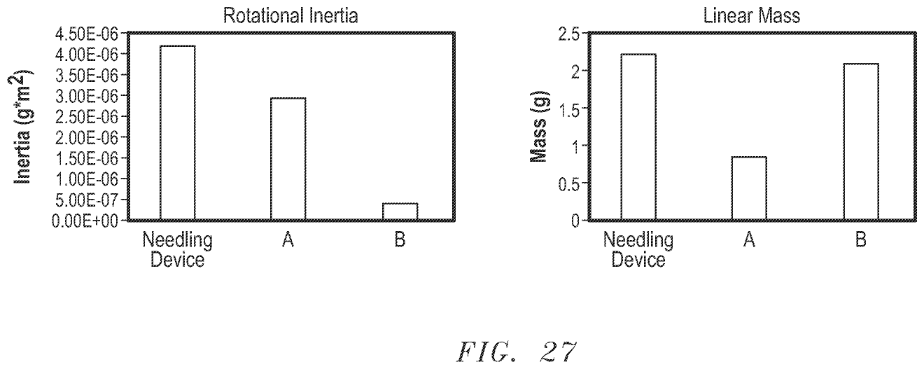

FIG. 27 is a graph illustrating the rotational inertia and linear mass of the motor linkages of FIG. 26. Referring to FIG. 27, the rotational inertia of a needling device according to an example of the present disclosure is approximately 4.19E-06 g*m² and is greater than the rotational inertia of other manufacturer devices. The linear mass of a needling device according to an example of the present disclosure is approximately 2.25 g and is greater than the linear mass of other manufacturer devices.

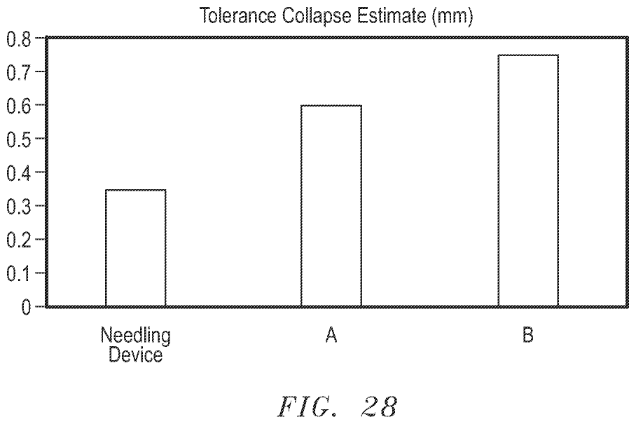

FIG. 28 is a graph illustrating the tolerance collapse of the motor linkages of FIG. 26. Linkage stiffness generally corresponds with the likelihood that the linkage mechanism would buckle at the moment that a needle strikes the skin and may depend on material stiffness and manufacturing tolerance collapse. With respect to manufacturing tolerance collapse, a clearance value between linkage components may be calculated to determine whether the mechanism buckles or crumples when loads are placed or the mechanism is locked in place (at the end of travel). Tolerance collapse is measured by measuring a difference in the position of the mechanism at the end of travel in a relaxed position versus the position of the mechanism at the end of travel in a compressed position. According to various examples, the tolerance collapse in a motor linkage of a needling device according to an example of the present disclosure may range from approximately 0.2 mm to approximately 0.5 mm. The tolerance collapse includes at least 0.2 mm, at least 0.3 mm, at least 0.4 mm, at least 0.5 mm, at most 0.2 mm, at most 0.3 mm, at most 0.4 mm, and at most 0.5 mm. In an example, the tolerance collapse may range from approximately 0.3 mm to approximately 0.4 mm. Still referring to FIG. 28, the tolerance collapse of a motor linkage of a needling device according to an example of the present disclosure is approximately 0.35 mm and is less than the tolerance collapse of other manufacturer devices.

With respect to materials used which impact the material stiffness and, in turn, the linkage stiffness, a motor linkage of a needling device according to an example of the present disclosure may be made of metals, polymers, glass, and combinations thereof. For example, the linkage is made of a combination of one or more of stainless steel, aluminum, PEEK, glass-filled polymer, and glass-filled metal.

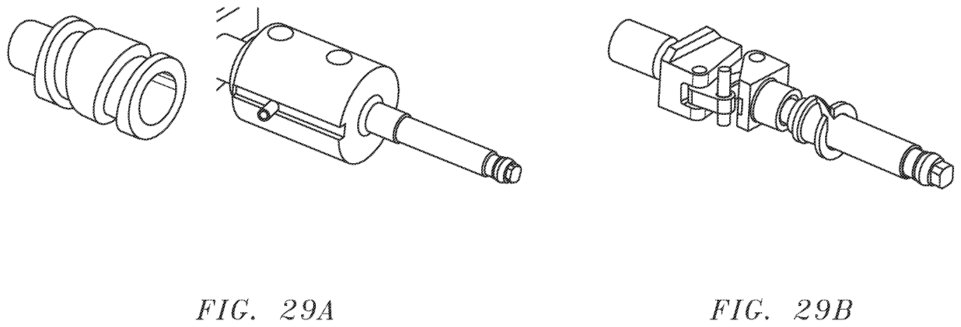

FIG. 29A is a diagram illustrating a schematic view of another example of a motor linkage of a needling device according to the present disclosure. Referring to FIG. 29A, a barrel cam motor linkage mechanism may be used with increased linkage mass and radius leading to a greatly increased rotational inertia. The materials, masses and tolerance collapse values described above may be used in the barrel cam linkage example leading to improved inertia and stiffness, as described above.

FIG. 29B is a diagram illustrating a schematic view of a further example of a motor linkage of a needling device according to the present disclosure. Referring to FIG. 29B, a scotch yoke motor linkage mechanism may be used with increased linkage mass and radius leading to a moderate increase in rotational inertia. The materials, masses and tolerance collapse values described above may be used in the scotch yoke cam linkage example leading to improved inertia and stiffness, as described above.

### 5.3 Depth Testing Verification

### 5.3.1 Needle Depth Measurement Study

A needle depth measurement tissue study has been conducted for the determination of needle depth precision during microneedling procedures using a needling device according to an example of the present disclosure. This study was completed with samples of porcine skin at targeted depths of 0.5 mm, 1.5 mm, and 2.0 mm. As an acceptance criteria, dye penetration associated with a needle track did not exceed targeted depth settings of the core.

Background. This study used excised porcine skin and contained only epidermis and dermis. All subcutaneous layers were removed by the skin supplier. The needling device according to an example of the present disclosure included a reusable cordless electromechanical core enclosed in a single-use disposable sterile sheath containing the needle array. The needles used in the needling device were solid core 32 gauge needles that do not cut tissue in the same manner as a hollow core needle. The solid core needles puncture the stratum corneum and epidermis during the microneedling procedure, separating elastin and collagen bundles of the dermis. This separation manifests as deformation and gaps among the collagen bundles of the dermis. In vivo, the viscoelastic properties of the tissue cause the dermis to recoil after needle retraction, making the needle penetration wound or “track” difficult to visualize in a simple H&E stain biopsy. As a result, observation and characterization of these needle punctures without additional visualization is difficult.

A Franz chamber-approach test fixture was developed to allow the sample to be pressurized post-needling to infuse dye into the needled tissue which remains stretched to prevent recoil of the tissue. The sample remained in radial tension throughout multiple experimental steps: microneedling, pressured infusion of pigment, rinsing, and application of fixative. A conservative, clinically relevant model was constructed to approximate conditions that could allow greatest needle penetration depth, including: the skin being stretched very taut, the subcutaneous fat being removed, and needling at a density relevant to clinical use.

Equipment and Materials. The study was conducted using a needling device according to an example of the present disclosure, including a needle dimension as described in Section 5.2.1, a needle array orientation as described in Section 5.2.2, and a motor and motor linkage as described in Section 5.2.3, and porcine skin. Post-procedure, all samples were prepared for histological analysis using H&E staining. The following equipment and materials were used:
Needling device core, adjustable from 0.5 to 2.5 mm target needle depth
Needling device sheaths (a single sheath was utilized for each sample)
SofTap pigment suspension, color 090 Charcoal (tattoo ink), SofTap Cosmetic Tattooing Supplies, as used by Sasaki.
Porcine skin samples, 1.5 mm thick, Stellen Medical Catalog No: 1-188 and/or USDA grade pork belly
Tupperware-type containers
Implemented concept of a Franz chamber pressure vessel and syringe
10% buffered formalin
DDS Digital Pathology System (or equivalent), slide annotation and analysis

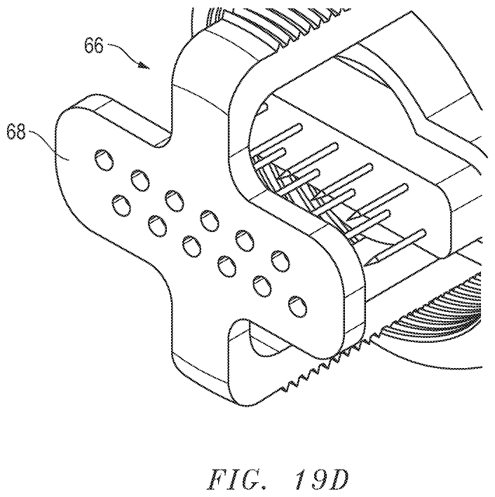

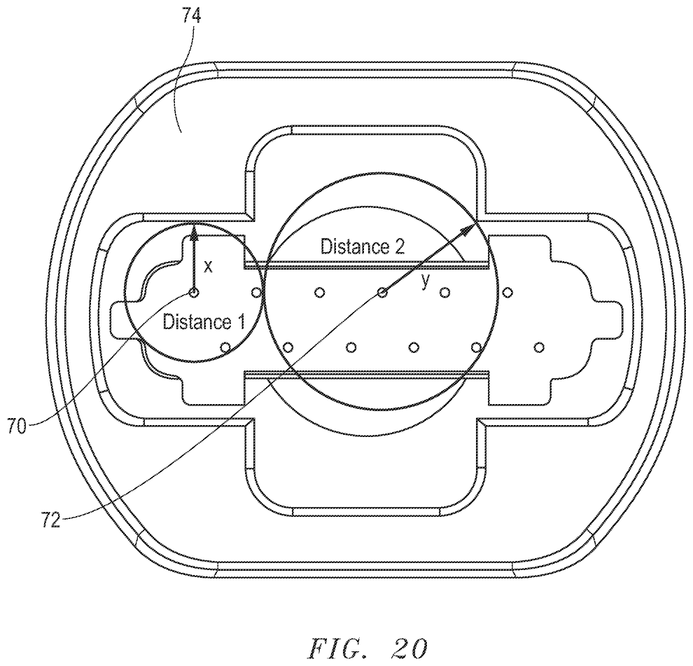

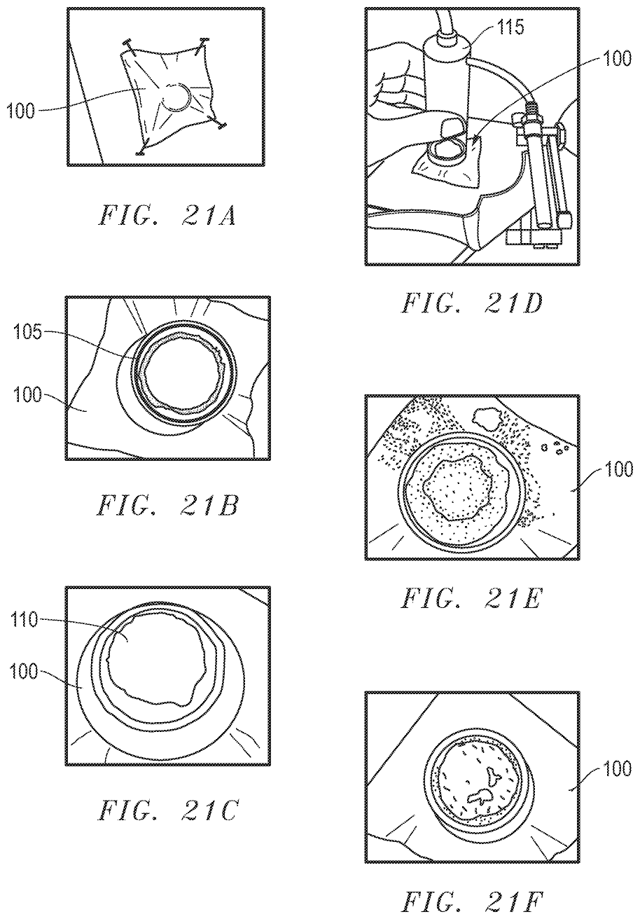

Testing Methodology. FIGS. 21A-21G are diagrams illustrating an example of a needle depth measurement tissue study. Referring to FIG. 21A, a porcine sample 100 is stretched over a rigid wire mesh flanged cylindrical shape with a flat top surface. Referring to FIG. 21B, a compression ring 105 is applied, and the sample is re-pinned in a maximum stretched position. A target needling zone mark is applied. Referring to FIG. 21C, 0.5 cc of diluted micropigmentation concentrate (SofTap 090 Charcoal) 110 is applied to a needled sample 100 (diluted to consistency of water). The full surface area of the sample 100 is needled with two overlapping passes (six passes overall, three each in perpendicular axes), at clinically relevant translation speed (2 cm/s). Referring to FIG. 21D, a pressure chamber 115 is placed over the sample 100. Referring to FIG. 21E, the pressure chamber 115 is pressurized to 13-15 psi for 30 seconds. Referring to FIG. 21F, the chamber 115 is removed and the sample 100 is washed and fixed in 10% buffered formalin. Referring to FIG. 21G, porcine tissue samples 100 are prepared for histology using H&E staining. Each sample 100 is prepared into four or five blocks of approximate equal size for interval depth sectioning.

Each tissue section is analyzed for (1) physical dimensions including maximum sample thickness and maximum sample length, (2) needle puncture count with physical puncture wounds being defined as any observation that penetrated the stratum corneum and punctured the epidermis, the measurement being made from the outer stratum corneum surface and all needle tracks being counted, and (3) needle track dye depth with the maximum depth of dye penetration being visible below each obvious needle track and the measurement being made from the outer stratum corneum surface. All histological sections are digitized for analysis. All measurements are made using the DDS slide imaging software provided by Mass Histology Service.

Results. Three tissue samples were evaluated, each sample being sectioned as illustrated in FIG. 21G. A single tissue section from each level was analyzed. The three tissue samples represented the 0.5 mm, 1.5 mm and 2.0 mm targeted depth setting of the core. The sheath test articles were inspected after use, without finding dislodged or bent needles. The following is a summary of the results:

TABLE 4
Dye Depth Measurements Summary Results
Summary Statistics of all Samples

| | 0.5 mm Depth Setting | 1.5 mm Depth Setting | 2.0 mm Depth Setting |
|---|---|---|---|
| | Sections L1-L4 | Sections L1-L5 | Sections L1-L5 |
| Number of Observed Needle Strikes | 209 | 135 | 125 |
| Number of Dye Penetration Measurments | 48 | 44 | 45 |
| Mean Sample Thickness, mm | 1.58 | 3.63 | 3.84 |
| Mean Sample Length, mm | 29.74 | 25.21 | 26.4 |
| Mean Dye Penetration Depth, mm | 0.191 | 0.577 | 0.758 |
| Std Dev, mm | 0.072 | 0.195 | 0.27 |
| Minimum Dye Penetration, mm | 0.061 | 0.009 | 0.331 |
| Maximum Dye Penetration, mm | 0.395 | 0.966 | 1.442 |

TABLE 5
Dye Depth Measurements Raw Histology Results

| 0.5 mm Needle | 1.5 mm Needle | 2.0 mm Needle |
|---|---|---|
| Depth Setting, um | Depth Setting, um | Depth Setting, um |
| 266.3 | 415 | 672.3 |
| 157.2 | 825.2 | 333.1 |
| 265.3 | 550.9 | 970.9 |
| 248.7 | 484.8 | 1322.6 |
| 226 | 234.5 | 1075.3 |
| 159.2 | 342.5 | 962.3 |
| 241.7 | 548.4 | 1309.5 |
| 190.3 | 423 | 439 |
| 232.6 | 555.9 | 628.7 |
| 232.9 | 633.6 | 1048.4 |
| 221.5 | 602.2 | 1138.3 |
| 89.6 | 760.4 | 870.3 |
| 124.6 | 754.6 | 741.1 |
| 132.5 | 552.9 | 558.8 |
| 361.8 | 373.1 | 719.4 |
| 292.4 | 717.1 | 648.3 |
| 201.5 | 568.5 | 615.8 |
| 81.2 | 573.7 | 661.4 |
| 143.4 | 651.1 | 663 |
| 139.2 | 461.5 | 883.9 |
| 120 | 397.2 | 513 |
| 167.1 | 524.8 | 674.3 |
| 292.3 | 329.3 | 792 |
| 246.7 | 661.9 | 710.6 |
| 182.7 | 390.2 | 430.8 |
| 130.5 | 427.5 | 485.4 |
| 164.2 | 769.3 | 746.1 |
| 66.9 | 399.2 | 748 |
| 194.2 | 714.5 | 522.5 |
| 221.5 | 403.2 | 1066 |
| 66.7 | 548.2 | 1174.6 |
| 202.8 | 738.8 | 444.8 |
| 163.5 | 839.8 | 452.6 |
| 108.6 | 796.5 | 561 |
| 132.2 | 518.2 | 738.3 |
| 282.2 | 654.6 | 468.4 |
| 267.8 | 470 | 512.1 |
| 218.7 | 631.4 | 910.7 |
| 61.3 | 599.6 | 954.1 |
| 394.6 | 960.3 | 854.2 |
| 194.4 | 913.6 | 1041.6 |
| 266.7 | 966 | 605.2 |
| 172.4 | 695.8 | 1442.4 |
| 179.2 | | 625.7 |
| 164.4 | | 382.1 |
| 148.1 | | |
| 183.3 | | |
| 160.8 | | |

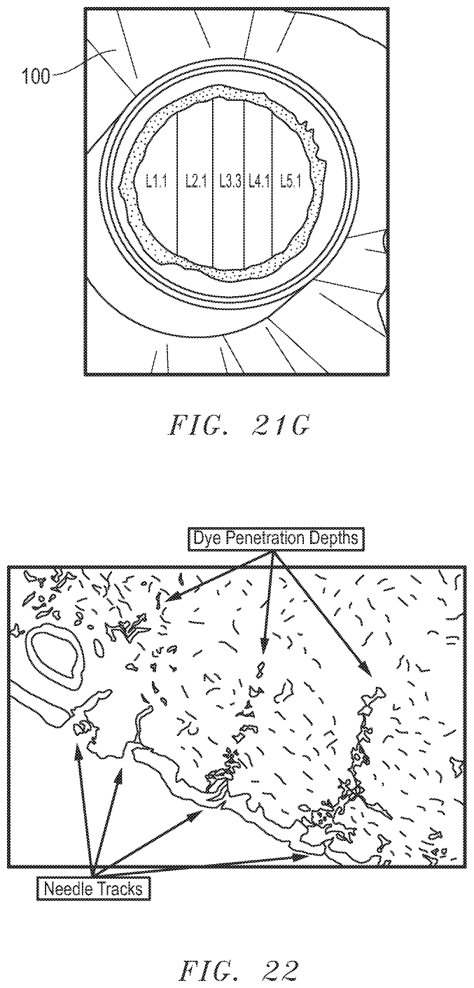

FIG. 22 is an annotated example of a single tissue section used for this study. Referring to FIG. 22, by the nature of the needling process and the orientation of microtome sectioning, the sections are not perfect cross-sectional cuts through needle tracks. Based on the high level of stretch applied to the sample and the use of a modified Franz chamber to infuse pigment into the needled tissue, this study represents a set of conditions most likely to show the possible penetration depth in clinical use of a needling device according to an example of the present disclosure.

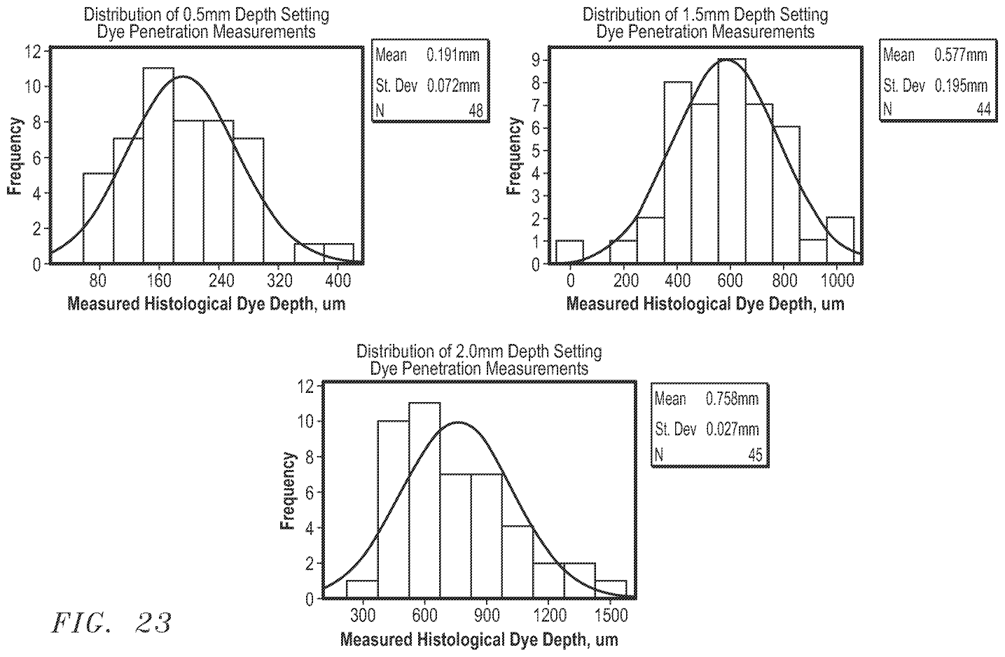

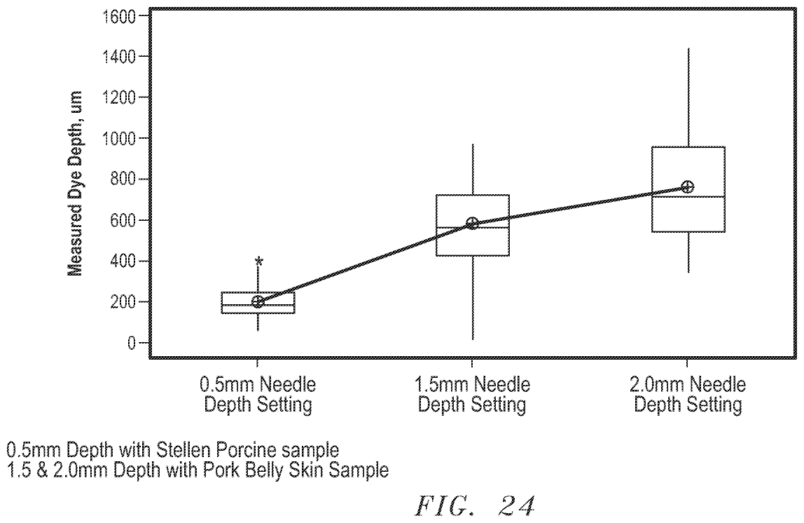

FIG. 23 shows histograms of the histologically measured dye penetration for the three different depth setting of the core. The stated acceptance criteria are that the dye penetration depth does not exceed the depth setting, and there are no instances of dye penetrating to depths greater than the depth setting of the core. Referring to FIG. 23, the dye penetration depth, as measured, is always less that the depth setting of the core. In no instance did dye penetration depth with associated needle track exceed the depth setting of the core. Accordingly, a needling device according to an example of the present disclosure does not pose any additional risk to a subject while in use.

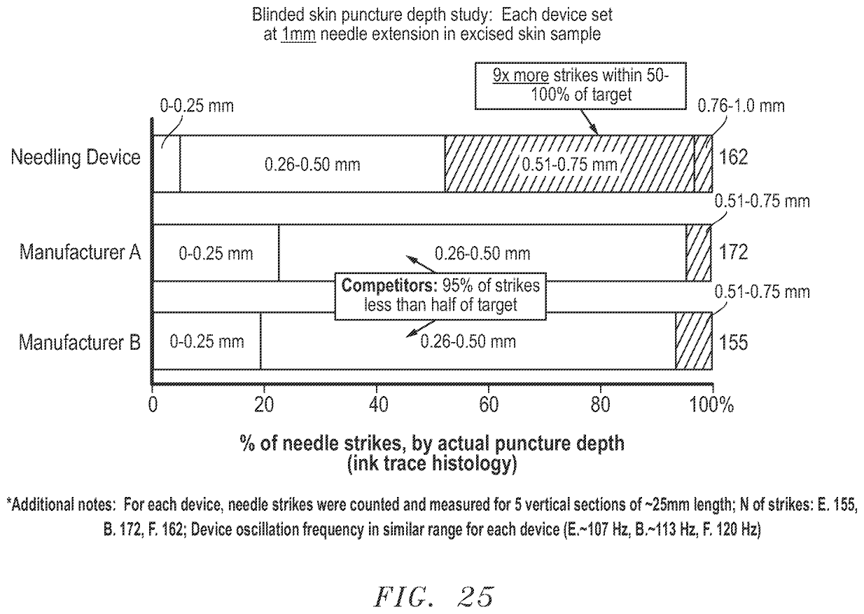

FIG. 24 illustrates that a needling device according to an example of the present disclosure functions as intended by the user depth setting. Referring to FIG. 24, a needling device according to an example of the present disclosure will have a tendency to provide deeper dye penetration with high depth setting. Nonetheless, a high degree of variation in depth is inherent to the tissue biology, the user, and processing of the sample.

### 5.3.2 Comparative Depth Measurement Study

Background. This study compared needle depths of a needling device according to an example of the present disclosure with other needling devices. The study was conducted using a needling device according to an example of the present disclosure, including a needle dimension as described in Section 5.2.1, a needle array orientation as described in Section 5.2.2, and a motor and motor linkage as described in Section 5.2.3, a needling device manufactured by manufacturer A, and a needling device manufactured by manufacturer B.

Testing Methodology. The surface area of three tissue samples were needled, one sample for each of the needling device of the present disclosure, manufacturer A's needling device, and manufacturer B's needling device. All needling devices were calibrated to needling at the 1.0 mm setting including the needling device of the present disclosure, manufacturer A's needling device, and manufacturer B's needling device. Twelve passes were applied through the dye (six passes in an east to west direction, and six passes in a north to south direction), at clinically relevant translation speed (2 cm/s). Pressure was applied post-needling at 5-10 psi for twenty seconds. The sample was removed from the fixture, washed and immersed in formalin.

Results. FIG. 25 is a diagram illustrating the results of an exemplary comparative needle depth study. Referring to FIG. 25, a percentage of needle strikes by actual puncture depth is illustrated for each of the needling device according to an example of the present disclosure, manufacturer A's needling device, and manufacturer B's needling device. For manufacturer A's device and manufacturer B's device, 95% of strikes had an actual depth ranging from 0 mm to 0.50 mm, or half of the target depth. For the needling device according to an example of the present disclosure, nine times more strikes had an actual depth ranging from 0.51 mm to 1.0 mm, or more than half of the target depth. For each device, needle strikes were counted and measured for five vertical sections of about 25 mm length. The number of strikes for manufacturer A's device was 155, for manufacturer B's device was 172, and for the needling device according to an example of the present disclosure was 162. The device oscillation frequency was in a similar range for all device (about 107 Hz for manufacturer A's device, about 113 Hz for manufacturer B's device, and about 120 Hz for the needling device according to an example of the present disclosure).

The following is a summary of the results of the study comparing manufacturer A's needling device, manufacturer B's needling device, and the needling device according to an example of the present disclosure. For brevity, manufacturer A's device is referred to as device A, manufacturer B's device is referred to as device B, and the needling device according to an example of the present disclosure is referred to as device X

TABLE 6
Comparative Strike Depth Results

| | | Strike | Strike | | | | Strike depth |
|---|---|---|---|---|---|---|---|
| Device | Slice | Num | ID | Histogram bucket 1 | Histogram bucket 2 | Histogram bucket 3 | (um) |
| A | L1 | 1 | GU-L1-1 | 351-400 um | 301-400 um | 26-50% of target depth | 356 |
| A | L1 | 2 | GU-L1-2 | 151-200 um | 101-200 um | 0-25% of target depth | 192 |
| A | L1 | 3 | GU-L1-3 | 301-350 um | 301-400 um | 26-50% of target depth | 319 |
| A | L1 | 4 | GU-L1-4 | 401-450 um | 401-500 um | 26-50% of target depth | 430 |
| A | L1 | 5 | GU-L1-5 | 351-400 um | 301-400 um | 26-50% of target depth | 394 |
| A | L1 | 6 | GU-L1-6 | 201-250 um | 201-300 um | 0-25% of target depth | 242 |
| A | L1 | 7 | GU-L1-7 | 501-550 um | 501-600 um | 51-75% of target depth | 522 |
| A | L1 | 8 | GU-L1-8 | 401-450 um | 401-500 um | 26-50% of target depth | 439 |
| A | L1 | 9 | GU-L1-9 | 401-450 um | 401-500 um | 26-50% of target depth | 414 |
| A | L1 | 10 | GU-L1-10 | 351-400 um | 301-400 um | 26-50% of target depth | 377 |
| A | L1 | 11 | GU-L1-11 | 351-400 um | 301-400 um | 26-50% of target depth | 389 |
| A | L1 | 12 | GU-L1-12 | 401-450 um | 401-500 um | 26-50% of target depth | 418 |
| A | L1 | 13 | GU-L1-13 | 351-400 um | 301-400 um | 26-50% of target depth | 354 |
| A | L1 | 14 | GU-L1-14 | 201-250 um | 201-300 um | 0-25% of target depth | 243 |
| A | L1 | 15 | GU-L1-15 | 151-200 um | 101-200 um | 0-25% of target depth | 183 |
| A | L1 | 16 | GU-L1-16 | 301-350 um | 301-400 um | 26-50% of target depth | 332 |
| A | L1 | 17 | GU-L1-17 | 401-450 um | 401-500 um | 26-50% of target depth | 416 |
| A | L1 | 18 | GU-L1-18 | 351-400 um | 301-400 um | 26-50% of target depth | 355 |
| A | L1 | 19 | GU-L1-19 | 251-300 um | 201-300 um | 26-50% of target depth | 281 |
| A | L1 | 20 | GU-L1-20 | 351-400 um | 301-400 um | 26-50% of target depth | 372 |
| A | L1 | 21 | GU-L1-21 | 201-250 um | 201-300 um | 0-25% of target depth | 214 |
| A | L1 | 22 | GU-L1-22 | 151-200 um | 101-200 um | 0-25% of target depth | 185 |
| A | L1 | 23 | GU-L1-23 | 301-350 um | 301-400 um | 26-50% of target depth | 331 |
| A | L1 | 24 | GU-L1-24 | 351-400 um | 301-400 um | 26-50% of target depth | 361 |
| A | L1 | 25 | GU-L1-25 | 301-350 um | 301-400 um | 26-50% of target depth | 309 |
| A | L1 | 26 | GU-L1-26 | 501-550 um | 501-600 um | 51-75% of target depth | 541 |
| A | L1 | 27 | GU-L1-27 | 301-350 um | 301-400 um | 26-50% of target depth | 302 |
| A | L1 | 28 | GU-L1-28 | 451-500 um | 401-500 um | 26-50% of target depth | 486 |
| A | L1 | 29 | GU-L1-29 | 351-400 um | 301-400 um | 26-50% of target depth | 377 |
| A | L1 | 30 | GU-L1-30 | 351-400 um | 301-400 um | 26-50% of target depth | 365 |
| A | L1 | 31 | GU-L1-31 | 451-500 um | 401-500 um | 26-50% of target depth | 496 |
| A | L1 | 32 | GU-L1-32 | 451-500 um | 401-500 um | 26-50% of target depth | 451 |
| A | L1 | 33 | GU-L1-33 | 301-350 um | 301-400 um | 26-50% of target depth | 340 |
| A | L2 | 34 | GU-L2-34 | 151-200 um | 101-200 um | 0-25% of target depth | 158 |
| A | L2 | 35 | GU-L2-35 | 401-450 um | 401-500 um | 26-50% of target depth | 411 |
| A | L2 | 36 | GU-L2-36 | 201-250 um | 201-300 um | 0-25% of target depth | 206 |
| A | L2 | 37 | GU-L2-37 | 301-350 um | 301-400 um | 26-50% of target depth | 336 |
| A | L2 | 38 | GU-L2-38 | 201-250 um | 201-300 um | 0-25% of target depth | 239 |
| A | L2 | 39 | GU-L2-39 | 351-400 um | 301-400 um | 26-50% of target depth | 353 |
| A | L2 | 40 | GU-L2-40 | 401-450 um | 401-500 um | 26-50% of target depth | 410 |
| A | L2 | 41 | GU-L2-41 | 351-400 um | 301-400 um | 26-50% of target depth | 385 |
| A | L2 | 42 | GU-L2-42 | 301-350 um | 301-400 um | 26-50% of target depth | 330 |
| A | L2 | 43 | GU-L2-43 | 301-350 um | 301-400 um | 26-50% of target depth | 341 |
| A | L2 | 44 | GU-L2-44 | 301-350 um | 301-400 um | 26-50% of target depth | 326 |
| A | L2 | 45 | GU-L2-45 | 351-400 um | 301-400 um | 26-50% of target depth | 351 |
| A | L2 | 46 | GU-L2-46 | 251-300 um | 201-300 um | 26-50% of target depth | 263 |
| A | L2 | 47 | GU-L2-47 | 201-250 um | 201-300 um | 0-25% of target depth | 232 |
| A | L2 | 48 | GU-L2-48 | 201-250 um | 201-300 um | 0-25% of target depth | 203 |
| A | L2 | 49 | GU-L2-49 | 201-250 um | 201-300 um | 0-25% of target depth | 233 |
| A | L2 | 50 | GU-L2-50 | 0-50 um | 0-100 um | 0-25% of target depth | 18 |
| A | L2 | 51 | GU-L2-51 | 251-300 um | 201-300 um | 26-50% of target depth | 272 |
| A | L2 | 52 | GU-L2-52 | 151-200 um | 101-200 um | 0-25% of target depth | 186 |
| A | L2 | 53 | GU-L2-53 | 301-350 um | 301-400 um | 26-50% of target depth | 330 |
| A | L2 | 54 | GU-L2-54 | 251-300 um | 201-300 um | 26-50% of target depth | 275 |
| A | L2 | 55 | GU-L2-55 | 251-300 um | 201-300 um | 26-50% of target depth | 285 |
| A | L2 | 56 | GU-L2-56 | 251-300 um | 201-300 um | 26-50% of target depth | 256 |
| A | L2 | 57 | GU-L2-57 | 151-200 um | 101-200 um | 0-25% of target depth | 184 |
| A | L2 | 58 | GU-L2-58 | 351-400 um | 301-400 um | 26-50% of target depth | 375 |
| A | L2 | 59 | GU-L2-59 | 351-400 um | 301-400 um | 26-50% of target depth | 350 |
| A | L2 | 60 | GU-L2-60 | 351-400 um | 301-400 um | 26-50% of target depth | 387 |
| A | L2 | 61 | GU-L2-61 | 351-400 um | 301-400 um | 26-50% of target depth | 366 |
| A | L2 | 62 | GU-L2-62 | 651-700 um | 601-700 um | 51-75% of target depth | 670 |
| A | L2 | 63 | GU-L2-63 | 251-300 um | 201-300 um | 26-50% of target depth | 256 |
| A | L2 | 64 | GU-L2-64 | 351-400 um | 301-400 um | 26-50% of target depth | 394 |
| A | L2 | 65 | GU-L2-65 | 251-300 um | 201-300 um | 26-50% of target depth | 262 |
| A | L2 | 66 | GU-L2-66 | 451-500 um | 401-500 um | 26-50% of target depth | 493 |
| A | L3 | 67 | GU-L3-67 | 101-150 um | 101-200 um | 0-25% of target depth | 124 |
| A | L3 | 68 | GU-L3-68 | 351-400 um | 301-400 um | 26-50% of target depth | 372 |
| A | L3 | 69 | GU-L3-69 | 301-350 um | 301-400 um | 26-50% of target depth | 306 |
| A | L3 | 70 | GU-L3-70 | 251-300 um | 201-300 um | 26-50% of target depth | 296 |
| A | L3 | 71 | GU-L3-71 | 251-300 um | 201-300 um | 26-50% of target depth | 290 |
| A | L3 | 72 | GU-L3-72 | 251-300 um | 201-300 um | 26-50% of target depth | 285 |
| A | L3 | 73 | GU-L3-73 | 251-300 um | 201-300 um | 26-50% of target depth | 261 |
| A | L3 | 74 | GU-L3-74 | 301-350 um | 301-400 um | 26-50% of target depth | 316 |
| A | L3 | 75 | GU-L3-75 | 451-500 um | 401-500 um | 26-50% of target depth | 471 |
| A | L3 | 76 | GU-L3-76 | 301-350 um | 301-400 um | 26-50% of target depth | 330 |
| A | L3 | 77 | GU-L3-77 | 301-350 um | 301-400 um | 26-50% of target depth | 323 |
| A | L3 | 78 | GU-L3-78 | 551-600 um | 501-600 um | 51-75% of target depth | 563 |
| A | L3 | 79 | GU-L3-79 | 351-400 um | 301-400 um | 26-50% of target depth | 387 |
| A | L3 | 80 | GU-L3-80 | 201-250 um | 201-300 um | 0-25% of target depth | 238 |
| A | L3 | 81 | GU-L3-81 | 501-550 um | 501-600 um | 51-75% of target depth | 520 |
| A | L3 | 82 | GU-L3-82 | 301-350 um | 301-400 um | 26-50% of target depth | 306 |
| A | L3 | 83 | GU-L3-83 | 501-550 um | 501-600 um | 51-75% of target depth | 512 |
| A | L3 | 84 | GU-L3-84 | 251-300 um | 201-300 um | 26-50% of target depth | 278 |
| A | L3 | 85 | GU-L3-85 | 251-300 um | 201-300 um | 26-50% of target depth | 270 |
| A | L3 | 86 | GU-L3-86 | 251-300 um | 201-300 um | 26-50% of target depth | 269 |
| A | L3 | 87 | GU-L3-87 | 451-500 um | 401-500 um | 26-50% of target depth | 499 |
| A | L3 | 88 | GU-L3-88 | 351-400 um | 301-400 um | 26-50% of target depth | 372 |
| A | L3 | 89 | GU-L3-89 | 351-400 um | 301-400 um | 26-50% of target depth | 398 |
| A | L3 | 90 | GU-L3-90 | 351-400 um | 301-400 um | 26-50% of target depth | 384 |
| A | L3 | 91 | GU-L3-91 | 251-300 um | 201-300 um | 26-50% of target depth | 291 |
| A | L3 | 92 | GU-L3-92 | 351-400 um | 301-400 um | 26-50% of target depth | 376 |
| A | L3 | 93 | GU-L3-93 | 401-450 um | 401-500 um | 26-50% of target depth | 436 |
| A | L3 | 94 | GU-L3-94 | 301-350 um | 301-400 um | 26-50% of target depth | 340 |
| A | L3 | 95 | GU-L3-95 | 351-400 um | 301-400 um | 26-50% of target depth | 363 |
| A | L3 | 96 | GU-L3-96 | 401-450 um | 401-500 um | 26-50% of target depth | 412 |
| A | L3 | 97 | GU-L3-97 | 301-350 um | 301-400 um | 26-50% of target depth | 342 |
| A | L3 | 98 | GU-L3-98 | 251-300 um | 201-300 um | 26-50% of target depth | 288 |
| A | L3 | 99 | GU-L3-99 | 301-350 um | 301-400 um | 26-50% of target depth | 323 |
| A | L3 | 100 | GU-L3-100 | 101-150 um | 101-200 um | 0-25% of target depth | 134 |
| A | L4 | 101 | GU-L4-101 | 351-400 um | 301-400 um | 26-50% of target depth | 362 |
| A | L4 | 102 | GU-L4-102 | 101-150 um | 101-200 um | 0-25% of target depth | 146 |
| A | L4 | 103 | GU-L4-103 | 501-550 um | 501-600 um | 51-75% of target depth | 546 |
| A | L4 | 104 | GU-L4-104 | 551-600 um | 501-600 um | 51-75% of target depth | 565 |
| A | L4 | 105 | GU-L4-105 | 401-450 um | 401-500 um | 26-50% of target depth | 403 |
| A | L4 | 106 | GU-L4-106 | 401-450 um | 401-500 um | 26-50% of target depth | 438 |
| A | L4 | 107 | GU-L4-107 | 351-400 um | 301-400 um | 26-50% of target depth | 368 |
| A | L4 | 108 | GU-L4-108 | 351-400 um | 301-400 um | 26-50% of target depth | 374 |
| A | L4 | 109 | GU-L4-109 | 251-300 um | 201-300 um | 26-50% of target depth | 279 |
| A | L4 | 110 | GU-L4-110 | 301-350 um | 301-400 um | 26-50% of target depth | 302 |
| A | L4 | 111 | GU-L4-111 | 251-300 um | 201-300 um | 26-50% of target depth | 261 |
| A | L4 | 112 | GU-L4-112 | 301-350 um | 301-400 um | 26-50% of target depth | 300 |
| A | L4 | 113 | GU-L4-113 | 301-350 um | 301-400 um | 26-50% of target depth | 341 |
| A | L4 | 114 | GU-L4-114 | 251-300 um | 201-300 um | 26-50% of target depth | 282 |
| A | L4 | 115 | GU-L4-115 | 251-300 um | 201-300 um | 26-50% of target depth | 297 |
| A | L4 | 116 | GU-L4-116 | 251-300 um | 201-300 um | 26-50% of target depth | 271 |
| A | L4 | 117 | GU-L4-117 | 101-150 um | 101-200 um | 0-25% of target depth | 132 |
| A | L4 | 118 | GU-L4-118 | 301-350 um | 301-400 um | 26-50% of target depth | 311 |
| A | L4 | 119 | GU-L4-119 | 201-250 um | 201-300 um | 0-25% of target depth | 240 |
| A | L4 | 120 | GU-L4-120 | 401-450 um | 401-500 um | 26-50% of target depth | 414 |
| A | L4 | 121 | GU-L4-121 | 251-300 um | 201-300 um | 26-50% of target depth | 281 |
| A | L4 | 122 | GU-L4-122 | 301-350 um | 301-400 um | 26-50% of target depth | 326 |
| A | L4 | 123 | GU-L4-123 | 151-200 um | 101-200 um | 0-25% of target depth | 187 |
| A | L4 | 124 | GU-L4-124 | 351-400 um | 301-400 um | 26-50% of target depth | 392 |
| A | L4 | 125 | GU-L4-125 | 551-600 um | 501-600 um | 51-75% of target depth | 564 |
| A | L4 | 126 | GU-L4-126 | 301-350 um | 301-400 um | 26-50% of target depth | 342 |
| A | L5 | 127 | GU-L5-127 | 51-100 um | 0-100 um | 0-25% of target depth | 80 |
| A | L5 | 128 | GU-L5-128 | 251-300 um | 201-300 um | 26-50% of target depth | 273 |
| A | L5 | 129 | GU-L5-129 | 401-450 um | 401-500 um | 26-50% of target depth | 437 |
| A | L5 | 130 | GU-L5-130 | 501-550 um | 501-600 um | 51-75% of target depth | 512 |
| A | L5 | 131 | GU-L5-131 | 451-500 um | 401-500 um | 26-50% of target depth | 452 |
| A | L5 | 132 | GU-L5-132 | 251-300 um | 201-300 um | 26-50% of target depth | 251 |
| A | L5 | 133 | GU-L5-133 | 401-450 um | 401-500 um | 26-50% of target depth | 420 |
| A | L5 | 134 | GU-L5-134 | 451-500 um | 401-500 um | 26-50% of target depth | 492 |
| A | L5 | 135 | GU-L5-135 | 301-350 um | 301-400 um | 26-50% of target depth | 348 |
| A | L5 | 136 | GU-L5-136 | 201-250 um | 201-300 um | 0-25% of target depth | 213 |
| A | L5 | 137 | GU-L5-137 | 101-150 um | 101-200 um | 0-25% of target depth | 124 |
| A | L5 | 138 | GU-L5-138 | 351-400 um | 301-400 um | 26-50% of target depth | 375 |
| A | L5 | 139 | GU-L5-139 | 301-350 um | 301-400 um | 26-50% of target depth | 340 |
| A | L5 | 140 | GU-L5-140 | 351-400 um | 301-400 um | 26-50% of target depth | 398 |
| A | L5 | 141 | GU-L5-141 | 351-400 um | 301-400 um | 26-50% of target depth | 354 |
| A | L5 | 142 | GU-L5-142 | 451-500 um | 401-500 um | 26-50% of target depth | 452 |
| A | L5 | 143 | GU-L5-143 | 351-400 um | 301-400 um | 26-50% of target depth | 391 |
| A | L5 | 144 | GU-L5-144 | 301-350 um | 301-400 um | 26-50% of target depth | 319 |
| A | L5 | 145 | GU-L5-145 | 251-300 um | 201-300 um | 26-50% of target depth | 270 |
| A | L5 | 146 | GU-L5-146 | 301-350 um | 301-400 um | 26-50% of target depth | 315 |
| A | L5 | 147 | GU-L5-147 | 251-300 um | 201-300 um | 26-50% of target depth | 276 |
| A | L5 | 148 | GU-L5-148 | 201-250 um | 201-300 um | 0-25% of target depth | 218 |
| A | L5 | 149 | GU-L5-149 | 201-250 um | 201-300 um | 0-25% of target depth | 239 |
| A | L5 | 150 | GU-L5-150 | 301-350 um | 301-400 um | 26-50% of target depth | 301 |
| A | L5 | 151 | GU-L5-151 | 351-400 um | 301-400 um | 26-50% of target depth | 391 |
| A | L5 | 152 | GU-L5-152 | 201-250 um | 201-300 um | 0-25% of target depth | 216 |
| A | L5 | 153 | GU-L5-153 | 401-450 um | 401-500 um | 26-50% of target depth | 442 |
| A | L5 | 154 | GU-L5-154 | 151-200 um | 101-200 um | 0-25% of target depth | 157 |
| A | L1 | 1 | UW-L1-1 | 101-150 um | 101-200 um | 0-25% of target depth | 132 |
| B | L1 | 2 | UW-L1-2 | 101-150 um | 101-200 um | 0-25% of target depth | 147 |
| B | L1 | 3 | UW-L1-3 | 251-300 um | 201-300 um | 26-50% of target depth | 284 |
| B | L1 | 4 | UW-L1-4 | 151-200 um | 101-200 um | 0-25% of target depth | 193 |
| B | L1 | 5 | UW-L1-5 | 101-150 um | 101-200 um | 0-25% of target depth | 119 |
| B | L1 | 6 | UW-L1-6 | 251-300 um | 201-300 um | 26-50% of target depth | 274 |
| B | L1 | 7 | UW-L1-7 | 351-400 um | 301-400 um | 26-50% of target depth | 389 |
| B | L1 | 8 | UW-L1-8 | 351-400 um | 301-400 um | 26-50% of target depth | 379 |
| B | L1 | 9 | UW-L1-9 | 401-450 um | 401-500 um | 26-50% of target depth | 416 |
| B | L1 | 10 | UW-L1-10 | 401-450 um | 401-500 um | 26-50% of target depth | 424 |
| B | L1 | 11 | UW-L1-11 | 401-450 um | 401-500 um | 26-50% of target depth | 428 |
| B | L1 | 12 | UW-L1-12 | 51-100 um | 0-100 um | 0-25% of target depth | 94 |
| B | L1 | 13 | UW-L1-13 | 101-150 um | 101-200 um | 0-25% of target depth | 123 |
| B | L1 | 14 | UW-L1-14 | 51-100 um | 0-100 um | 0-25% of target depth | 96 |
| B | L1 | 15 | UW-L1-15 | 401-450 um | 401-500 um | 26-50% of target depth | 426 |
| B | L1 | 16 | UW-L1-16 | 501-550 um | 501-600 um | 51-75% of target depth | 544 |
| B | L1 | 17 | UW-L1-17 | 501-550 um | 501-600 um | 51-75% of target depth | 513 |
| B | L1 | 18 | UW-L1-18 | 451-500 um | 401-500 um | 26-50% of target depth | 456 |
| B | L1 | 19 | UW-L1-19 | 401-450 um | 401-500 um | 26-50% of target depth | 439 |
| B | L1 | 20 | UW-L1-20 | 151-200 um | 101-200 um | 0-25% of target depth | 150 |
| B | L1 | 21 | UW-L1-21 | 351-400 um | 301-400 um | 26-50% of target depth | 366 |
| B | L1 | 22 | UW-L1-22 | 301-350 um | 301-400 um | 26-50% of target depth | 323 |
| B | L1 | 23 | UW-L1-23 | 251-300 um | 201-300 um | 26-50% of target depth | 276 |
| B | L1 | 24 | UW-L1-24 | 251-300 um | 201-300 um | 26-50% of target depth | 290 |
| B | L1 | 25 | UW-L1-25 | 451-500 um | 401-500 um | 26-50% of target depth | 482 |
| B | L1 | 26 | UW-L1-26 | 351-400 um | 301-400 um | 26-50% of target depth | 362 |
| B | L1 | 27 | UW-L1-27 | 151-200 um | 101-200 um | 0-25% of target depth | 174 |
| B | L1 | 28 | UW-L1-28 | 101-150 um | 101-200 um | 0-25% of target depth | 134 |
| B | L1 | 29 | UW-L1-29 | 551-600 um | 501-600 um | 51-75% of target depth | 572 |
| B | L1 | 30 | UW-L1-30 | 201-250 um | 201-300 um | 0-25% of target depth | 242 |
| B | L1 | 31 | UW-L1-31 | 251-300 um | 201-300 um | 26-50% of target depth | 281 |
| B | L1 | 32 | UW-L1-32 | 401-450 um | 401-500 um | 26-50% of target depth | 416 |
| B | L1 | 33 | UW-L1-33 | 401-450 um | 401-500 um | 26-50% of target depth | 425 |
| B | L1 | 34 | UW-L1-34 | 351-400 um | 301-400 um | 26-50% of target depth | 381 |
| B | L1 | 35 | UW-L1-35 | 601-650 um | 601-700 um | 51-75% of target depth | 642 |
| B | L1 | 36 | UW-L1-36 | 101-150 um | 101-200 um | 0-25% of target depth | 143 |
| B | L1 | 37 | UW-L1-37 | 151-200 um | 101-200 um | 0-25% of target depth | 192 |
| B | L1 | 38 | UW-L1-38 | 151-200 um | 101-200 um | 0-25% of target depth | 172 |
| B | L1 | 39 | UW-L1-39 | 201-250 um | 201-300 um | 0-25% of target depth | 228 |
| B | L1 | 40 | UW-L1-40 | 151-200 um | 101-200 um | 0-25% of target depth | 176 |
| B | L2 | 41 | UW-L2-41 | 201-250 um | 201-300 um | 0-25% of target depth | 216 |
| B | L2 | 42 | UW-L2-42 | 351-400 um | 301-400 um | 26-50% of target depth | 379 |
| B | L2 | 43 | UW-L2-43 | 401-450 um | 401-500 um | 26-50% of target depth | 423 |
| B | L2 | 44 | UW-L2-44 | 301-350 um | 301-400 um | 26-50% of target depth | 305 |
| B | L2 | 45 | UW-L2-45 | 351-400 um | 301-400 um | 26-50% of target depth | 356 |
| B | L2 | 46 | UW-L2-46 | 401-450 um | 401-500 um | 26-50% of target depth | 410 |
| B | L2 | 47 | UW-L2-47 | 401-450 um | 401-500 um | 26-50% of target depth | 446 |
| B | L2 | 48 | UW-L2-48 | 451-500 um | 401-500 um | 26-50% of target depth | 476 |
| B | L2 | 49 | UW-L2-49 | 251-300 um | 201-300 um | 26-50% of target depth | 253 |
| B | L2 | 50 | UW-L2-50 | 301-350 um | 301-400 um | 26-50% of target depth | 346 |
| B | L2 | 51 | UW-L2-51 | 351-400 um | 301-400 um | 26-50% of target depth | 353 |
| B | L2 | 52 | UW-L2-52 | 301-350 um | 301-400 um | 26-50% of target depth | 316 |
| B | L2 | 53 | UW-L2-53 | 301-350 um | 301-400 um | 26-50% of target depth | 301 |
| B | L2 | 54 | UW-L2-54 | 451-500 um | 401-500 um | 26-50% of target depth | 463 |
| B | L2 | 55 | UW-L2-55 | 251-300 um | 201-300 um | 26-50% of target depth | 253 |
| B | L2 | 56 | UW-L2-56 | 451-500 um | 401-500 um | 26-50% of target depth | 498 |
| B | L2 | 57 | UW-L2-57 | 451-500 um | 401-500 um | 26-50% of target depth | 491 |
| B | L2 | 58 | UW-L2-58 | 401-450 um | 401-500 um | 26-50% of target depth | 405 |
| B | L2 | 59 | UW-L2-59 | 401-450 um | 401-500 um | 26-50% of target depth | 412 |
| B | L2 | 60 | UW-L2-60 | 351-400 um | 301-400 um | 26-50% of target depth | 372 |
| B | L2 | 61 | UW-L2-61 | 351-400 um | 301-400 um | 26-50% of target depth | 392 |
| B | L2 | 62 | UW-L2-62 | 201-250 um | 201-300 um | 0-25% of target depth | 238 |
| B | L2 | 63 | UW-L2-63 | 251-300 um | 201-300 um | 26-50% of target depth | 250 |
| B | L2 | 64 | UW-L2-64 | 151-200 um | 101-200 um | 0-25% of target depth | 165 |
| B | L2 | 65 | UW-L2-65 | 501-550 um | 501-600 um | 51-75% of target depth | 546 |
| B | L2 | 66 | UW-L2-66 | 401-450 um | 401-500 um | 26-50% of target depth | 403 |
| B | L2 | 67 | UW-L2-67 | 201-250 um | 201-300 um | 0-25% of target depth | 200 |
| B | L2 | 68 | UW-L2-68 | 401-450 um | 401-500 um | 26-50% of target depth | 416 |
| B | L2 | 69 | UW-L2-69 | 601-650 um | 601-700 um | 51-75% of target depth | 645 |
| B | L2 | 70 | UW-L2-70 | 401-450 um | 401-500 um | 26-50% of target depth | 423 |
| B | L2 | 71 | UW-L2-71 | 401-450 um | 401-500 um | 26-50% of target depth | 424 |
| B | L2 | 72 | UW-L2-72 | 451-500 um | 401-500 um | 26-50% of target depth | 487 |
| B | L3 | 73 | UW-L3-73 | 301-350 um | 301-400 um | 26-50% of target depth | 347 |
| B | L3 | 74 | UW-L3-74 | 351-400 um | 301-400 um | 26-50% of target depth | 374 |
| B | L3 | 75 | UW-L3-75 | 151-200 um | 101-200 um | 0-25% of target depth | 191 |
| B | L3 | 76 | UW-L3-76 | 451-500 um | 401-500 um | 26-50% of target depth | 470 |
| B | L3 | 77 | UW-L3-77 | 251-300 um | 201-300 um | 26-50% of target depth | 264 |
| B | L3 | 78 | UW-L3-78 | 301-350 um | 301-400 um | 26-50% of target depth | 302 |
| B | L3 | 79 | UW-L3-79 | 251-300 um | 201-300 um | 26-50% of target depth | 279 |
| B | L3 | 80 | UW-L3-80 | 251-300 um | 201-300 um | 26-50% of target depth | 274 |
| B | L3 | 81 | UW-L3-81 | 251-300 um | 201-300 um | 26-50% of target depth | 271 |
| B | L3 | 82 | UW-L3-82 | 201-250 um | 201-300 um | 0-25% of target depth | 218 |
| B | L3 | 83 | UW-L3-83 | 201-250 um | 201-300 um | 0-25% of target depth | 228 |
| B | L3 | 84 | UW-L3-84 | 201-250 um | 201-300 um | 0-25% of target depth | 249 |
| B | L3 | 85 | UW-L3-85 | 251-300 um | 201-300 um | 26-50% of target depth | 293 |
| B | L3 | 86 | UW-L3-86 | 151-200 um | 101-200 um | 0-25% of target depth | 174 |
| B | L3 | 87 | UW-L3-87 | 151-200 um | 101-200 um | 0-25% of target depth | 186 |
| B | L3 | 88 | UW-L3-88 | 151-200 um | 101-200 um | 0-25% of target depth | 150 |
| B | L3 | 89 | UW-L3-89 | 201-250 um | 201-300 um | 0-25% of target depth | 238 |
| B | L3 | 90 | UW-L3-90 | 251-300 um | 201-300 um | 26-50% of target depth | 284 |
| B | L3 | 91 | UW-L3-91 | 201-250 um | 201-300 um | 0-25% of target depth | 204 |
| B | L3 | 92 | UW-L3-92 | 401-450 um | 401-500 um | 26-50% of target depth | 412 |
| B | L3 | 93 | UW-L3-93 | 201-250 um | 201-300 um | 0-25% of target depth | 241 |
| B | L3 | 94 | UW-L3-94 | 301-350 um | 301-400 um | 26-50% of target depth | 324 |
| B | L3 | 95 | UW-L3-95 | 251-300 um | 201-300 um | 26-50% of target depth | 285 |
| B | L3 | 96 | UW-L3-96 | 301-350 um | 301-400 um | 26-50% of target depth | 329 |
| B | L3 | 97 | UW-L3-97 | 351-400 um | 301-400 um | 26-50% of target depth | 386 |
| B | L3 | 98 | UW-L3-98 | 401-450 um | 401-500 um | 26-50% of target depth | 414 |
| B | L3 | 99 | UW-L3-99 | 351-400 um | 301-400 um | 26-50% of target depth | 351 |
| B | L3 | 100 | UW-L3-100 | 301-350 um | 301-400 um | 26-50% of target depth | 307 |
| B | L3 | 101 | UW-L3-101 | 401-450 um | 401-500 um | 26-50% of target depth | 419 |
| B | L3 | 102 | UW-L3-102 | 301-350 um | 301-400 um | 26-50% of target depth | 316 |
| B | L3 | 103 | UW-L3-103 | 251-300 um | 201-300 um | 26-50% of target depth | 258 |
| B | L3 | 104 | UW-L3-104 | 401-450 um | 401-500 um | 26-50% of target depth | 422 |
| B | L3 | 105 | UW-L3-105 | 501-550 um | 501-600 um | 51-75% of target depth | 509 |
| B | L3 | 106 | UW-L3-106 | 401-450 um | 401-500 um | 26-50% of target depth | 438 |
| B | L3 | 107 | UW-L3-107 | 301-350 um | 301-400 um | 26-50% of target depth | 317 |
| B | L3 | 108 | UW-L3-108 | 351-400 um | 301-400 um | 26-50% of target depth | 383 |
| B | L3 | 109 | UW-L3-109 | 301-350 um | 301-400 um | 26-50% of target depth | 302 |
| B | L3 | 110 | UW-L3-110 | 351-400 um | 301-400 um | 26-50% of target depth | 353 |
| B | L3 | 111 | UW-L3-111 | 301-350 um | 301-400 um | 26-50% of target depth | 332 |
| B | L3 | 112 | UW-L3-112 | 401-450 um | 401-500 um | 26-50% of target depth | 411 |
| B | L3 | 113 | UW-L3-113 | 251-300 um | 201-300 um | 26-50% of target depth | 294 |
| B | L3 | 114 | UW-L3-114 | 201-250 um | 201-300 um | 0-25% of target depth | 222 |
| B | L4 | 115 | UW-L4-115 | 201-250 um | 201-300 um | 0-25% of target depth | 220 |
| B | L4 | 116 | UW-L4-116 | 451-500 um | 401-500 um | 26-50% of target depth | 458 |
| B | L4 | 117 | UW-L4-117 | 301-350 um | 301-400 um | 26-50% of target depth | 347 |
| B | L4 | 118 | UW-L4-118 | 251-300 um | 201-300 um | 26-50% of target depth | 290 |
| B | L4 | 119 | UW-L4-119 | 251-300 um | 201-300 um | 26-50% of target depth | 285 |
| B | L4 | 120 | UW-L4-120 | 451-500 um | 401-500 um | 26-50% of target depth | 465 |
| B | L4 | 121 | UW-L4-121 | 351-400 um | 301-400 um | 26-50% of target depth | 365 |
| B | L4 | 122 | UW-L4-122 | 251-300 um | 201-300 um | 26-50% of target depth | 267 |
| B | L4 | 123 | UW-L4-123 | 301-350 um | 301-400 um | 26-50% of target depth | 345 |
| B | L4 | 124 | UW-L4-124 | 251-300 um | 201-300 um | 26-50% of target depth | 279 |
| B | L4 | 125 | UW-L4-125 | 151-200 um | 101-200 um | 0-25% of target depth | 192 |
| B | L4 | 126 | UW-L4-126 | 151-200 um | 101-200 um | 0-25% of target depth | 169 |
| B | L4 | 127 | UW-L4-127 | 251-300 um | 201-300 um | 26-50% of target depth | 285 |
| B | L4 | 128 | UW-L4-128 | 251-300 um | 201-300 um | 26-50% of target depth | 290 |
| B | L4 | 129 | UW-L4-129 | 351-400 um | 301-400 um | 26-50% of target depth | 352 |
| B | L4 | 130 | UW-L4-130 | 351-400 um | 301-400 um | 26-50% of target depth | 371 |
| B | L4 | 131 | UW-L4-131 | 151-200 um | 101-200 um | 0-25% of target depth | 153 |
| B | L4 | 132 | UW-L4-132 | 401-450 um | 401-500 um | 26-50% of target depth | 434 |
| B | L4 | 133 | UW-L4-133 | 401-450 um | 401-500 um | 26-50% of target depth | 429 |
| B | L4 | 134 | UW-L4-134 | 451-500 um | 401-500 um | 26-50% of target depth | 479 |
| B | L4 | 135 | UW-L4-135 | 401-450 um | 401-500 um | 26-50% of target depth | 437 |
| B | L4 | 136 | UW-L4-136 | 451-500 um | 401-500 um | 26-50% of target depth | 489 |
| B | L4 | 137 | UW-L4-137 | 251-300 um | 201-300 um | 26-50% of target depth | 292 |
| B | L4 | 138 | UW-L4-138 | 351-400 um | 301-400 um | 26-50% of target depth | 384 |
| B | L4 | 139 | UW-L4-139 | 451-500 um | 401-500 um | 26-50% of target depth | 466 |
| B | L4 | 140 | UW-L4-140 | 401-450 um | 401-500 um | 26-50% of target depth | 401 |
| B | L4 | 141 | UW-L4-141 | 301-350 um | 301-400 um | 26-50% of target depth | 341 |
| B | L4 | 142 | UW-L4-142 | 351-400 um | 301-400 um | 26-50% of target depth | 365 |
| B | L4 | 143 | UW-L4-143 | 201-250 um | 201-300 um | 0-25% of target depth | 243 |
| B | L4 | 144 | UW-L4-144 | 251-300 um | 201-300 um | 26-50% of target depth | 275 |
| B | L4 | 145 | UW-L4-145 | 401-450 um | 401-500 um | 26-50% of target depth | 407 |
| B | L4 | 146 | UW-L4-146 | 501-550 um | 501-600 um | 51-75% of target depth | 522 |
| B | L5 | 147 | UW-L5-147 | 401-450 um | 401-500 um | 26-50% of target depth | 415 |
| B | L5 | 148 | UW-L5-148 | 401-450 um | 401-500 um | 26-50% of target depth | 403 |
| B | L5 | 149 | UW-L5-149 | 151-200 um | 101-200 um | 0-25% of target depth | 173 |
| B | L5 | 150 | UW-L5-150 | 251-300 um | 201-300 um | 26-50% of target depth | 290 |
| B | L5 | 151 | UW-L5-151 | 251-300 um | 201-300 um | 26-50% of target depth | 293 |
| B | L5 | 152 | UW-L5-152 | 251-300 um | 201-300 um | 26-50% of target depth | 266 |
| B | L5 | 153 | UW-L5-153 | 301-350 um | 301-400 um | 26-50% of target depth | 320 |
| B | L5 | 154 | UW-L5-154 | 401-450 um | 401-500 um | 26-50% of target depth | 401 |
| B | L5 | 155 | UW-L5-155 | 201-250 um | 201-300 um | 0-25% of target depth | 201 |
| B | L5 | 156 | UW-L5-156 | 101-150 um | 101-200 um | 0-25% of target depth | 142 |
| B | L5 | 157 | UW-L5-157 | 251-300 um | 201-300 um | 26-50% of target depth | 252 |
| B | L5 | 158 | UW-L5-158 | 301-350 um | 301-400 um | 26-50% of target depth | 342 |
| B | L5 | 159 | UW-L5-159 | 301-350 um | 301-400 um | 26-50% of target depth | 345 |
| B | L5 | 160 | UW-L5-160 | 301-350 um | 301-400 um | 26-50% of target depth | 327 |
| B | L5 | 161 | UW-L5-161 | 451-500 um | 401-500 um | 26-50% of target depth | 462 |
| B | L5 | 162 | UW-L5-162 | 301-350 um | 301-400 um | 26-50% of target depth | 335 |
| B | L5 | 163 | UW-L5-163 | 301-350 um | 301-400 um | 26-50% of target depth | 328 |
| B | L5 | 164 | UW-L5-164 | 251-300 um | 201-300 um | 26-50% of target depth | 276 |
| B | L5 | 165 | UW-L5-165 | 301-350 um | 301-400 um | 26-50% of target depth | 340 |
| B | L5 | 166 | UW-L5-166 | 351-400 um | 301-400 um | 26-50% of target depth | 368 |
| B | L5 | 167 | UW-L5-167 | 301-350 um | 301-400 um | 26-50% of target depth | 315 |
| B | L5 | 168 | UW-L5-168 | 451-500 um | 401-500 um | 26-50% of target depth | 470 |
| B | L5 | 169 | UW-L5-169 | 301-350 um | 301-400 um | 26-50% of target depth | 349 |
| B | L5 | 170 | UW-L5-170 | 401-450 um | 401-500 um | 26-50% of target depth | 440 |
| B | L5 | 171 | UW-L5-171 | 401-450 um | 401-500 um | 26-50% of target depth | 423 |
| B | L5 | 172 | UW-L5-172 | 201-250 um | 201-300 um | 0-25% of target depth | 240 |
| B | L5 | 173 | UW-L5-173 | 301-350 um | 301-400 um | 26-50% of target depth | 327 |
| X | L1 | 1 | CE-L1-1 | 351-400 um | 301-400 um | 26-50% of target depth | 386 |
| X | L1 | 2 | CE-L1-2 | 551-600 um | 501-600 um | 51-75% of target depth | 592 |
| X | L1 | 3 | CE-L1-3 | 401-450 um | 401-500 um | 26-50% of target depth | 425 |
| X | L1 | 4 | CE-L1-4 | 701-750 um | 701-800 um | 51-75% of target depth | 747 |
| X | L1 | 5 | CE-L1-5 | 501-550 um | 501-600 um | 51-75% of target depth | 500 |
| X | L1 | 6 | CE-L1-6 | 601-650 um | 601-700 um | 51-75% of target depth | 623 |
| X | L1 | 7 | CE-L1-7 | 501-550 um | 501-600 um | 51-75% of target depth | 545 |
| X | L1 | 8 | CE-L1-8 | 451-500 um | 401-500 um | 26-50% of target depth | 486 |
| X | L1 | 9 | CE-L1-9 | 501-550 um | 501-600 um | 51-75% of target depth | 512 |
| X | L1 | 10 | CE-L1-10 | 601-650 um | 601-700 um | 51-75% of target depth | 612 |
| X | L1 | 11 | CE-L1-11 | 501-550 um | 501-600 um | 51-75% of target depth | 540 |
| X | L1 | 12 | CE-L1-12 | 651-700 um | 601-700 um | 51-75% of target depth | 688 |
| X | L1 | 13 | CE-L1-13 | 551-600 um | 501-600 um | 51-75% of target depth | 554 |
| X | L1 | 14 | CE-L1-14 | 501-550 um | 501-600 um | 51-75% of target depth | 538 |
| X | L1 | 15 | CE-L1-15 | 551-600 um | 501-600 um | 51-75% of target depth | 589 |
| X | L1 | 16 | CE-L1-16 | 551-600 um | 501-600 um | 51-75% of target depth | 566 |
| X | L1 | 17 | CE-L1-17 | 601-650 um | 601-700 um | 51-75% of target depth | 618 |
| X | L1 | 18 | CE-L1-18 | 501-550 um | 501-600 um | 51-75% of target depth | 517 |
| X | L1 | 19 | CE-L1-19 | 601-650 um | 601-700 um | 51-75% of target depth | 642 |
| X | L1 | 20 | CE-L1-20 | 651-700 um | 601-700 um | 51-75% of target depth | 696 |
| X | L1 | 21 | CE-L1-21 | 551-600 um | 501-600 um | 51-75% of target depth | 553 |
| X | L1 | 22 | CE-L1-22 | 451-500 um | 401-500 um | 26-50% of target depth | 477 |
| X | L1 | 23 | CE-L1-23 | 401-450 um | 401-500 um | 26-50% of target depth | 431 |
| X | L1 | 24 | CE-L1-24 | 701-750 um | 701-800 um | 51-75% of target depth | 741 |
| X | L1 | 25 | CE-L1-25 | 501-550 um | 501-600 um | 51-75% of target depth | 519 |
| X | L1 | 26 | CE-L1-26 | 551-600 um | 501-600 um | 51-75% of target depth | 557 |
| X | L1 | 27 | CE-L1-27 | 451-500 um | 401-500 um | 26-50% of target depth | 484 |
| X | L1 | 28 | CE-L1-28 | 451-500 um | 401-500 um | 26-50% of target depth | 473 |
| X | L1 | 29 | CE-L1-29 | 451-500 um | 401-500 um | 26-50% of target depth | 474 |
| X | L1 | 30 | CE-L1-30 | 451-500 um | 401-500 um | 26-50% of target depth | 452 |
| X | L1 | 31 | CE-L1-31 | 501-550 um | 501-600 um | 51-75% of target depth | 541 |
| X | L1 | 32 | CE-L1-32 | 751+ um | 701-800 um | 76-100% of target depth | 766 |
| X | L1 | 33 | CE-L1-33 | 401-450 um | 401-500 um | 26-50% of target depth | 423 |
| X | L1 | 34 | CE-L1-34 | 651-700 um | 601-700 um | 51-75% of target depth | 692 |
| X | L1 | 35 | CE-L1-35 | 351-400 um | 301-400 um | 26-50% of target depth | 396 |
| X | L1 | 36 | CE-L1-36 | 601-650 um | 601-700 um | 51-75% of target depth | 639 |
| X | L1 | 37 | CE-L1-37 | 651-700 um | 601-700 um | 51-75% of target depth | 698 |
| X | L1 | 38 | CE-L1-38 | 501-550 um | 501-600 um | 51-75% of target depth | 512 |
| X | L1 | 39 | CE-L1-39 | 401-450 um | 401-500 um | 26-50% of target depth | 412 |
| X | L1 | 40 | CE-L1-40 | 601-650 um | 601-700 um | 51-75% of target depth | 644 |
| X | L1 | 41 | CE-L1-41 | 351-400 um | 301-400 um | 26-50% of target depth | 360 |
| X | L1 | 42 | CE-L1-42 | 251-300 um | 201-300 um | 26-50% of target depth | 272 |
| X | L1 | 43 | CE-L1-43 | 551-600 um | 501-600 um | 51-75% of target depth | 567 |
| X | L1 | 44 | CE-L1-44 | 351-400 um | 301-400 um | 26-50% of target depth | 372 |
| X | L1 | 45 | CE-L1-45 | 301-350 um | 301-400 um | 26-50% of target depth | 324 |
| X | L1 | 46 | CE-L1-46 | 401-450 um | 401-500 um | 26-50% of target depth | 420 |
| X | L2 | 47 | CE-L2-47 | 251-300 um | 201-300 um | 26-50% of target depth | 279 |
| X | L2 | 48 | CE-L2-48 | 451-500 um | 401-500 um | 26-50% of target depth | 470 |
| X | L2 | 49 | CE-L2-49 | 651-700 um | 601-700 um | 51-75% of target depth | 695 |
| X | L2 | 50 | CE-L2-50 | 551-600 um | 501-600 um | 51-75% of target depth | 551 |
| X | L2 | 51 | CE-L2-51 | 351-400 um | 301-400 um | 26-50% of target depth | 367 |
| X | L2 | 52 | CE-L2-52 | 751+ um | 701-800 um | 76-100% of target depth | 758 |
| X | L2 | 53 | CE-L2-53 | 501-550 um | 501-600 um | 51-75% of target depth | 528 |
| X | L2 | 54 | CE-L2-54 | 201-250 um | 201-300 um | 0-25% of target depth | 249 |
| X | L2 | 55 | CE-L2-55 | 351-400 um | 301-400 um | 26-50% of target depth | 397 |
| X | L2 | 56 | CE-L2-56 | 401-450 um | 401-500 um | 26-50% of target depth | 446 |
| X | L2 | 57 | CE-L2-57 | 351-400 um | 301-400 um | 26-50% of target depth | 371 |
| X | L2 | 58 | CE-L2-58 | 251-300 um | 201-300 um | 26-50% of target depth | 294 |
| X | L2 | 59 | CE-L2-59 | 601-650 um | 601-700 um | 51-75% of target depth | 630 |
| X | L2 | 60 | CE-L2-60 | 601-650 um | 601-700 um | 51-75% of target depth | 610 |
| X | L2 | 61 | CE-L2-61 | 501-550 um | 501-600 um | 51-75% of target depth | 525 |
| X | L2 | 62 | CE-L2-62 | 701-750 um | 701-800 um | 51-75% of target depth | 724 |
| X | L2 | 63 | CE-L2-63 | 551-600 um | 501-600 um | 51-75% of target depth | 576 |
| X | L2 | 64 | CE-L2-64 | 451-500 um | 401-500 um | 26-50% of target depth | 496 |
| X | L2 | 65 | CE-L2-65 | 651-700 um | 601-700 um | 51-75% of target depth | 656 |
| X | L2 | 66 | CE-L2-66 | 601-650 um | 601-700 um | 51-75% of target depth | 608 |
| X | L2 | 67 | CE-L2-67 | 451-500 um | 401-500 um | 26-50% of target depth | 454 |
| X | L2 | 68 | CE-L2-68 | 651-700 um | 601-700 um | 51-75% of target depth | 666 |
| X | L2 | 69 | CE-L2-69 | 501-550 um | 501-600 um | 51-75% of target depth | 523 |
| X | L2 | 70 | CE-L2-70 | 301-350 um | 301-400 um | 26-50% of target depth | 345 |
| X | L2 | 71 | CE-L2-71 | 501-550 um | 501-600 um | 51-75% of target depth | 534 |
| X | L2 | 72 | CE-L2-72 | 401-450 um | 401-500 um | 26-50% of target depth | 439 |
| X | L2 | 73 | CE-L2-73 | 301-350 um | 301-400 um | 26-50% of target depth | 319 |
| X | L2 | 74 | CE-L2-74 | 401-450 um | 401-500 um | 26-50% of target depth | 432 |
| X | L2 | 75 | CE-L2-75 | 551-600 um | 501-600 um | 51-75% of target depth | 588 |
| X | L2 | 76 | CE-L2-76 | 501-550 um | 501-600 um | 51-75% of target depth | 521 |
| X | L2 | 77 | CE-L2-77 | 601-650 um | 601-700 um | 51-75% of target depth | 601 |
| X | L2 | 78 | CE-L2-78 | 651-700 um | 601-700 um | 51-75% of target depth | 698 |
| X | L2 | 79 | CE-L2-79 | 501-550 um | 501-600 um | 51-75% of target depth | 544 |
| X | L2 | 80 | CE-L2-80 | 351-400 um | 301-400 um | 26-50% of target depth | 393 |
| X | L2 | 81 | CE-L2-81 | 251-300 um | 201-300 um | 26-50% of target depth | 269 |
| X | L2 | 82 | CE-L2-82 | 551-600 um | 501-600 um | 51-75% of target depth | 564 |
| X | L2 | 83 | CE-L2-83 | 551-600 um | 501-600 um | 51-75% of target depth | 575 |
| X | L2 | 84 | CE-L2-84 | 351-400 um | 301-400 um | 26-50% of target depth | 393 |
| X | L2 | 85 | CE-L2-85 | 301-350 um | 301-400 um | 26-50% of target depth | 324 |
| X | L2 | 86 | CE-L2-86 | 201-250 um | 201-300 um | 0-25% of target depth | 225 |
| X | L2 | 87 | CE-L2-87 | 301-350 um | 301-400 um | 26-50% of target depth | 327 |
| X | L2 | 88 | CE-L2-88 | 301-350 um | 301-400 um | 26-50% of target depth | 324 |
| X | L2 | 89 | CE-L2-89 | 351-400 um | 301-400 um | 26-50% of target depth | 365 |
| X | L2 | 90 | CE-L2-90 | 451-500 um | 401-500 um | 26-50% of target depth | 491 |
| X | L2 | 91 | CE-L2-91 | 351-400 um | 301-400 um | 26-50% of target depth | 372 |
| X | L2 | 92 | CE-L2-92 | 151-200 um | 101-200 um | 0-25% of target depth | 181 |
| X | L3 | 93 | CE-L3-93 | 301-350 um | 301-400 um | 26-50% of target depth | 309 |
| X | L3 | 94 | CE-L3-94 | 251-300 um | 201-300 um | 26-50% of target depth | 287 |
| X | L3 | 95 | CE-L3-95 | 351-400 um | 301-400 um | 26-50% of target depth | 391 |
| X | L3 | 96 | CE-L3-96 | 601-650 um | 601-700 um | 51-75% of target depth | 601 |
| X | L3 | 97 | CE-L3-97 | 501-550 um | 501-600 um | 51-75% of target depth | 517 |
| X | L3 | 98 | CE-L3-98 | 501-550 um | 501-600 um | 51-75% of target depth | 514 |
| X | L3 | 99 | CE-L3-99 | 451-500 um | 401-500 um | 26-50% of target depth | 487 |
| X | L3 | 100 | CE-L3-100 | 401-450 um | 401-500 um | 26-50% of target depth | 425 |
| X | L3 | 101 | CE-L3-101 | 301-350 um | 301-400 um | 26-50% of target depth | 314 |
| X | L3 | 102 | CE-L3-102 | 251-300 um | 201-300 um | 26-50% of target depth | 281 |
| X | L3 | 103 | CE-L3-103 | 251-300 um | 201-300 um | 26-50% of target depth | 296 |
| X | L3 | 104 | CE-L3-104 | 551-600 um | 501-600 um | 51-75% of target depth | 597 |
| X | L3 | 105 | CE-L3-105 | 551-600 um | 501-600 um | 51-75% of target depth | 576 |
| X | L3 | 106 | CE-L3-106 | 351-400 um | 301-400 um | 26-50% of target depth | 368 |
| X | L3 | 107 | CE-L3-107 | 501-550 um | 501-600 um | 51-75% of target depth | 500 |
| X | L3 | 108 | CE-L3-108 | 351-400 um | 301-400 um | 26-50% of target depth | 371 |
| X | L3 | 109 | CE-L3-109 | 451-500 um | 401-500 um | 26-50% of target depth | 476 |
| X | L3 | 110 | CE-L3-110 | 601-650 um | 601-700 um | 51-75% of target depth | 642 |
| X | L3 | 111 | CE-L3-111 | 351-400 um | 301-400 um | 26-50% of target depth | 387 |
| X | L3 | 112 | CE-L3-112 | 401-450 um | 401-500 um | 26-50% of target depth | 435 |
| X | L3 | 113 | CE-L3-113 | 301-350 um | 301-400 um | 26-50% of target depth | 323 |
| X | L3 | 114 | CE-L3-114 | 251-300 um | 201-300 um | 26-50% of target depth | 287 |
| X | L3 | 115 | CE-L3-115 | 251-300 um | 201-300 um | 26-50% of target depth | 262 |
| X | L3 | 116 | CE-L3-116 | 351-400 um | 301-400 um | 26-50% of target depth | 385 |
| X | L3 | 117 | CE-L3-117 | 301-350 um | 301-400 um | 26-50% of target depth | 339 |
| X | L3 | 118 | CE-L3-118 | 601-650 um | 601-700 um | 51-75% of target depth | 646 |
| X | L3 | 119 | CE-L3-119 | 301-350 um | 301-400 um | 26-50% of target depth | 327 |
| X | L3 | 120 | CE-L3-120 | 551-600 um | 501-600 um | 51-75% of target depth | 555 |
| X | L3 | 121 | CE-L3-121 | 301-350 um | 301-400 um | 26-50% of target depth | 340 |
| X | L3 | 122 | CE-L3-122 | 201-250 um | 201-300 um | 0-25% of target depth | 220 |
| X | L3 | 123 | CE-L3-123 | 401-450 um | 401-500 um | 26-50% of target depth | 429 |
| X | L3 | 124 | CE-L3-124 | 301-350 um | 301-400 um | 26-50% of target depth | 324 |
| X | L3 | 125 | CE-L3-125 | 301-350 um | 301-400 um | 26-50% of target depth | 307 |
| X | L3 | 126 | CE-L3-126 | 301-350 um | 301-400 um | 26-50% of target depth | 317 |
| X | L3 | 127 | CE-L3-127 | 301-350 um | 301-400 um | 26-50% of target depth | 307 |
| X | L3 | 128 | CE-L3-128 | 351-400 um | 301-400 um | 26-50% of target depth | 381 |
| X | L3 | 129 | CE-L3-129 | 301-350 um | 301-400 um | 26-50% of target depth | 324 |
| X | L3 | 130 | CE-L3-130 | 301-350 um | 301-400 um | 26-50% of target depth | 340 |
| X | L3 | 131 | CE-L3-131 | 701-750 um | 701-800 um | 51-75% of target depth | 722 |
| X | L3 | 132 | CE-L3-132 | 351-400 um | 301-400 um | 26-50% of target depth | 367 |
| X | L4 | 133 | CE-L4-133 | 501-550 um | 501-600 um | 51-75% of target depth | 523 |
| X | L4 | 134 | CE-L4-134 | 601-650 um | 601-700 um | 51-75% of target depth | 647 |
| X | L4 | 135 | CE-L4-135 | 301-350 um | 301-400 um | 26-50% of target depth | 339 |
| X | L4 | 136 | CE-L4-136 | 501-550 um | 501-600 um | 51-75% of target depth | 511 |
| X | L4 | 137 | CE-L4-137 | 451-500 um | 401-500 um | 26-50% of target depth | 465 |
| X | L4 | 138 | CE-L4-138 | 551-600 um | 501-600 um | 51-75% of target depth | 556 |
| X | L4 | 139 | CE-L4-139 | 451-500 um | 401-500 um | 26-50% of target depth | 468 |
| X | L4 | 140 | CE-L4-140 | 451-500 um | 401-500 um | 26-50% of target depth | 460 |
| X | L4 | 141 | CE-L4-141 | 201-250 um | 201-300 um | 0-25% of target depth | 248 |
| X | L4 | 142 | CE-L4-142 | 201-250 um | 201-300 um | 0-25% of target depth | 220 |
| X | L4 | 143 | CE-L4-143 | 101-150 um | 101-200 um | 0-25% of target depth | 132 |
| X | L4 | 144 | CE-L4-144 | 351-400 um | 301-400 um | 26-50% of target depth | 355 |
| X | L5 | 145 | CE-L5-145 | 551-600 um | 501-600 um | 51-75% of target depth | 597 |
| X | L5 | 146 | CE-L5-146 | 601-650 um | 601-700 um | 51-75% of target depth | 630 |
| X | L5 | 147 | CE-L5-147 | 751+ um | 701-800 um | 76-100% of target depth | 764 |
| X | L5 | 148 | CE-L5-148 | 601-650 um | 601-700 um | 51-75% of target depth | 638 |
| X | L5 | 149 | CE-L5-149 | 751+ um | 701-800 um | 76-100% of target depth | 763 |
| X | L5 | 150 | CE-L5-150 | 551-600 um | 501-600 um | 51-75% of target depth | 583 |
| X | L5 | 151 | CE-L5-151 | 351-400 um | 301-400 um | 26-50% of target depth | 383 |
| X | L5 | 152 | CE-L5-152 | 351-400 um | 301-400 um | 26-50% of target depth | 382 |
| X | L5 | 153 | CE-L5-153 | 551-600 um | 501-600 um | 51-75% of target depth | 566 |
| X | L5 | 154 | CE-L5-154 | 551-600 um | 501-600 um | 51-75% of target depth | 594 |
| X | L5 | 155 | CE-L5-155 | 401-450 um | 401-500 um | 26-50% of target depth | 440 |
| X | L5 | 156 | CE-L5-156 | 551-600 um | 501-600 um | 51-75% of target depth | 571 |
| X | L5 | 157 | CE-L5-157 | 551-600 um | 501-600 um | 51-75% of target depth | 595 |
| X | L5 | 158 | CE-L5-158 | 551-600 um | 501-600 um | 51-75% of target depth | 550 |
| X | L5 | 159 | CE-L5-159 | 751+ um | 801-900 um | >100% | 843 |
| X | L5 | 160 | CE-L5-160 | 201-250 um | 201-300 um | 0-25% of target depth | 243 |
| X | L5 | 161 | CE-L5-161 | 701-750 um | 701-800 um | 51-75% of target depth | 707 |
| X | L5 | 162 | CE-L5-162 | 301-350 um | 301-400 um | 26-50% of target depth | 331 |

TABLE 7
Estimated Strikes Per Slice

| | Device X | Device B | Device A | Unit |
|---|---|---|---|---|
| Passes | 12 | 12 | 12 | passes |
| Number of needles | 12 | 14 | 12 | needles |
| Oscillation frequency | 120 | 113 | 107 | l/s |
| Speed of travel | 20 | 20 | 20 | mm/s |
| Average sample length | 26.08 | 23.28 | 21.2 | mm |
| Trace width | 0.07 | 0.07 | 0.07 | mm |
| Average sample area | 1.8256 | 1.6296 | 1.484 | mm² |
| Average total area | 434 | 434 | 434 | mm² |
| Coverage % | 0.4202% | 0.3751% | 0.3416% | % of sample |
| Needle strikes per second | 1440 | 1582 | 1284 | strikes/s |
| Estimated pass length average | 20 | 20 | 20 | mm |
| Estimated treatment time | 12 | 12 | 12 | s |
| Total needle strikes | 17280 | 18984 | 15408 | strikes per sample |
| Estimated theoretical strikes per slice | 72.6 | 71.2 | 52.6 | strikes |
| Actual strikes per slice | 32.4 | 34.6 | 30.8 | strikes |
| % detection | 45% | 49% | 59% | |

TABLE 8
Depth Realization Comparison

| | Values | | | |
|---|---|---|---|---|
| | Average of | Count of | Max of | StdDev of |
| Device | Strike depth | Strike depth | Strike depth | Strike depth |
| A | 332 | 155 | 670 | 106 |
| B | 333 | 172 | 645 | 109 |
| X | 479 | 162 | 843 | 145 |
| Grand Total | 381 | 489 | 843 | 139 |

TABLE 9
Detailed Histogram Results A

| | | | | |
|---|---|---|---|---|
| Count of Strike depth | Device | | | Grand |
| Histogram bucket | A | B | X | Total |

| | | | | | |
|---|---|---|---|---|---|
| 0-50 | um | 1 | | | 1 |
| 51-100 | um | 1 | 2 | | 3 |
| 101-150 | um | 6 | 6 | 1 | 13 |
| 151-200 | um | 8 | 15 | 1 | 24 |
| 201-250 | um | 14 | 16 | 6 | 36 |
| 251-300 | um | 27 | 30 | 9 | 66 |
| 301-350 | um | 30 | 27 | 20 | 77 |
| 351-400 | um | 34 | 21 | 21 | 76 |
| 401-450 | um | 15 | 32 | 12 | 59 |
| 451-500 | um | 9 | 15 | 15 | 39 |
| 501-550 | um | 6 | 5 | 20 | 31 |
| 551-600 | um | 3 | 1 | 23 | 27 |
| 601-650 | um | | 2 | 16 | 18 |
| 651-700 | um | 1 | | 8 | 9 |
| 701-750 | um | | | 5 | 5 |
| 751+ | um | | | 5 | 5 |

| | | | | |
|---|---|---|---|---|
| Grand Total | 155 | 172 | 162 | 489 |

TABLE 10
Detailed Histogram Results B

| Count of Strike depth | Device | | | Grand |
|---|---|---|---|---|
| Histogram bucket 2 | A | B | X | Total |
| 0-100 um | 1.29% | 1.16% | 0.00% | 0.82% |
| 101-200 um | 9.03% | 12.21% | 1.23% | 7.57% |
| 201-300 um | 26.45% | 26.74% | 9.26% | 20.86% |
| 301-400 um | 41.29% | 27.91% | 25.31% | 31.29% |
| 401-500 um | 15.48% | 27.33% | 16.67% | 20.04% |
| 501-600 um | 5.81% | 3.49% | 26.54% | 11.86% |
| 601-700 um | 0.65% | 1.16% | 14.81% | 5.52% |
| 701-800 um | 0.00% | 0.00% | 5.56% | 1.84% |
| 801-900 um | 0.00% | 0.00% | 0.62% | 0.20% |
| Grand Total | 100.00% | 100.00% | 100.00% | 100.00% |

TABLE 11
Simple Histogram Results

| Count of Strike depth | Device | | | Grand |
|---|---|---|---|---|
| Histogram bucket3 | A | B | X | Total |
| 0-25% of target depth | 19.35% | 22.67% | 4.94% | 15.75% |
| 26-50% of target depth | 74.19% | 72.67% | 47.53% | 64.83% |
| 51-75% of target depth | 6.45% | 4.65% | 44.44% | 18.40% |
| 76-100% of target depth | 0.00% | 0.00% | 2.47% | 0.82% |
| >100% | 0.00% | 0.00% | 0.62% | 0.20% |
| Grand Total | 100.00% | 100.00% | 100.00% | 100.00% |

The favorable depth verification results as describe above are attributed to the improved structural features described throughout the present disclosure. All tests yielding favorable results were performed using a needling device according to an example of the present disclosure, including a needle dimension as described in Section 5.2.1, a needle array orientation as described in Section 5.2.2, and a motor and motor linkage as described in Section 5.2.3.

It will be appreciated by those skilled in the art that changes could be made to the embodiments described above without departing from the broad inventive concept thereof. It is understood, therefore, that the invention disclosed herein is not limited to the particular embodiments disclosed, but it is intended to cover modifications within the spirit and scope of the present invention as defined by the appended claims.

What is claimed is:

1. A needling device, comprising:
a plurality of needles forming a needle array; and
a motor assembly for driving the needle array,
wherein each of the plurality of needles comprises a needle tip at one end, and tapers at a taper angle and along a taper length to a maximum needle diameter at the other end, and
wherein the maximum needle diameter ranges from approximately 0.20 mm to approximately 0.24 mm.

2. The needling device of claim 1, wherein the taper length ranges from approximately 1 mm to approximately 2 mm.

3. The needling device of any one of the preceding claims, wherein the taper angle ranges from approximately 5 degrees to approximately 15 degrees.

4. The needling device of any one of the preceding claims, wherein the needle tip has a tip radius ranging from approximately 0.015 mm to approximately 0.025 mm.

5. The needling device of 1 any one of the preceding claims, wherein, in use, a skin reference surface of the needling device is in contact with a subject's skin, and the skin reference surface has a surface area ranging from approximately 45 mm² to approximately 105 mm².

6. The needling device of any one of the preceding claims, wherein, in use, a skin reference surface of the needling device is in contact with a subject's skin, and an average distance between each needle of the plurality of needles of the needle array and the skin reference surface ranges from approximately 0.10 mm to approximately 2.5 mm.

7. The needling device of claim 6, wherein a distance between each needle of the plurality of needles of the needle array and the skin reference surface is the same for all needles.

8. The needling device of claim 6, wherein a first distance between one of the plurality of needles of the needle array and the skin reference surface is different than a second distance between another of the plurality of needles of the needle array and the skin reference surface.

9. The needling device of any one of the preceding claims, wherein the motor assembly comprises a motor linkage, and wherein the motor linkage comprises a rotational component with a total mass ranging from approximately 0.5 grams to approximately 35 grams and a rotational radius ranging from approximately 1.5 mm to approximately 3.5 mm, and the motor linkage comprises a linear component with a total mass ranging from approximately 1.5 grams to approximately 3.5 grams.

10. The needling device of any one of the preceding claims, wherein an actual penetration depth of the plurality of needles into a subject's skin does not exceed a depth setting on the needling device.

11. The needling device of any one of the preceding claims, wherein a mean value of an actual penetration depth of the plurality of needles is at least 0.2 mm in response to a depth setting of the needling device of 0.5 mm, at least 0.6 mm in response to a depth setting of the needling device of 1.5 mm, and at least 0.75 mm in response to a depth setting of the needling device of 2.0 mm.

12. The needling device of any one of the preceding claims, wherein, in use, an actual penetration depth of the plurality of needles is at least 50% of a target depth based on a depth setting of the device for at least 45% of all needle strikes of the plurality of needles.

13. The needling device of any one of the preceding claims, wherein, in use, an actual penetration depth of the plurality of needles is at least 50% of a target depth based on a depth setting of the device for at least 35% of all needle strikes of the plurality of needles.

14. The needling device of any one of the preceding claims, wherein, in use, an actual penetration depth of the plurality of needles is at least 50% of a target depth based on a depth setting of the device for at least 25% of all needle strikes of the plurality of needles.

15. The needling device of any one of the preceding claims, wherein, in use, an actual penetration depth of the plurality of needles is at least 50% of a target depth based on a depth setting of the device for at least 15% of all needle strikes of the plurality of needles.

16. The needling device of any one of the preceding claims, further comprising a sheath assembly comprising the needle array and a main unit comprising the motor assembly.

17. A needling device, comprising:
a plurality of needles forming a needle array; and
a motor assembly for driving the needle array,
wherein each of the plurality of needles comprises a needle tip at one end, and tapers at a taper angle and along a taper length to a maximum needle diameter at the other end, and
wherein the taper length ranges from approximately 1 mm to approximately 2 mm.

18. The needling device of claim 17, wherein the taper angle ranges from approximately 5 degrees to approximately 15 degrees.

19. The needling device of any one of claim 17 or 18, wherein the needle tip has a tip radius ranging from approximately 0.015 mm to approximately 0.025 mm.

20. The needling device of any one of claims 17 to 19, wherein, in use, a skin reference surface of the needling device is in contact with a subject's skin, and the skin reference surface has a surface area ranging from approximately 45 mm² to approximately 105 mm².

21. The needling device of any one of claims 17 to 20, wherein, in use, a skin reference surface of the needling device is in contact with a subject's skin, and an average distance between each needle of the plurality of needles of the needle array and the skin reference surface area ranges from approximately 0.10 mm to approximately 2.5 mm.

22. The needling device of claim 21, wherein a distance between each needle of the plurality of needles of the needle array and the skin reference surface is the same for all needles.

23. The needling device of claim 21, wherein a first distance between one of the plurality of needles of the needle array and the skin reference surface is different than a second distance between another of the plurality of needles of the needle array and the skin reference surface.

24. The needling device of any one of claims 17 to 23, wherein the motor assembly comprises a motor linkage, and wherein the motor linkage comprises a rotational component with a total mass ranging from approximately 0.5 grams to approximately 35 grams and a rotational radius ranging from approximately 1.5 mm to approximately 3.5 mm, and the motor linkage comprises a linear component with a total mass ranging from approximately 1.5 grams to approximately 3.5 grams.

25. The needling device of any one of claims 17 to 24, wherein an actual penetration depth of the plurality of needles into a subject's skin does not exceed a depth setting of the needling device.

26. The needling device of any one of claims 17 to 25, wherein a mean value of an actual penetration depth of the plurality of needles is at least 0.2 mm in response to a depth setting of the needling device of 0.5 mm, at least 0.6 mm in response to a depth setting of the needling device of 1.5 mm, and at least 0.75 mm in response to a depth setting of the needling device of 2.0 mm.

27. The needling device of any one of claims 17 to 26, wherein, in use, an actual penetration depth of the plurality of needles is at least 50% of a target depth based on a depth setting of the device for at least 45% of all needle strikes of the plurality of needles.

28. The needling device of any one of claims 17 to 27, wherein, in use, an actual penetration depth of the plurality of needles is at least 50% of a target depth based on a depth setting of the device for at least 35% of all needle strikes of the plurality of needles.

29. The needling device of any one of claims 17 to 28, wherein, in use, an actual penetration depth of the plurality of needles is at least 50% of a target depth based on a depth setting of the device for at least 25% of all needle strikes of the plurality of needles.

30. The needling device of any one of claims 17 to 29, wherein, in use, an actual penetration depth of the plurality of needles is at least 50% of a target depth based on a depth setting of the device for at least 15% of all needle strikes of the plurality of needles.

31. The needling device of any one of claims 17 to 30, further comprising a sheath assembly comprising the needle array and a main unit comprising the motor assembly.

32. A needling device, comprising:
a plurality of needles forming a needle array; and
a motor assembly for driving the needle array,
wherein each of the plurality of needles comprises a needle tip at one end, and tapers at a taper angle and along a taper length to a maximum needle diameter at the other end, and
wherein the taper angle ranges from approximately 5 degrees to approximately 15 degrees.

33. The needling device of claim 32, wherein the needle tip has a tip radius ranging from approximately 0.015 mm to approximately 0.025 mm.

34. The needling device of any one of claim 32 or 33, wherein, in use, a skin reference surface of the needling device is in contact with a subject's skin, and the skin reference surface has a surface area ranging from approximately 45 mm² to approximately 105 mm².

35. The needling device of any one of claims 32 to 34, wherein, in use, a skin reference surface of the needling device is in contact with a subject's skin, and an average distance between each needle of the plurality of needles of the needle array and the skin reference surface area ranges from approximately 0.10 mm to approximately 2.5 mm.

36. The needling device of claim 35, wherein a distance between each needle of the plurality of needles of the needle array and the skin reference surface is the same for all needles.

37. The needling device of claim 35, wherein a first distance between one of the plurality of needles of the needle array and the skin reference surface is different than a second distance between another of the plurality of needles of the needle array and the skin reference surface.

38. The needling device of any one of claims 32 to 27, wherein the motor assembly comprises a motor linkage, and wherein the motor linkage comprises a rotational component with a total mass ranging from approximately 0.5 grams to approximately 35 grams and a rotational radius ranging from approximately 1.5 mm to approximately 3.5 mm, and the motor linkage comprises a linear component with a total mass ranging from approximately 1.5 grams to approximately 3.5 grams.

39. The needling device of any one of claims 32 to 38, wherein an actual penetration depth of the plurality of needles into a subject's skin does not exceed a depth setting of the needling device.

40. The needling device of any one of claims 32 to 39, wherein a mean value of an actual penetration depth of the plurality of needles is at least 0.2 mm in response to a depth setting of the needling device of 0.5 mm, at least 0.6 mm in response to a depth setting of the needling device of 1.5 mm, and at least 0.75 mm in response to a depth setting of the needling device of 2.0 mm.

41. The needling device of any one of claims 32 to 40, wherein, in use, an actual penetration depth of the plurality of needles is at least 50% of a target depth based on a depth setting of the device for at least 45% of all needle strikes of the plurality of needles.

42. The needling device of any one of claims 32 to 41, wherein, in use, an actual penetration depth of the plurality of needles is at least 50% of a target depth based on a depth setting of the device for at least 35% of all needle strikes of the plurality of needles.

43. The needling device of any one of claims 32 to 42, wherein, in use, an actual penetration depth of the plurality of needles is at least 50% of a target depth based on a depth setting of the device for at least 25% of all needle strikes of the plurality of needles.

44. The needling device of any one of claims 32 to 43, wherein, in use, an actual penetration depth of the plurality of needles is at least 50% of a target depth based on a depth setting of the device for at least 15% of all needle strikes of the plurality of needles.

45. The needling device of any one of claims 32 to 44, further comprising a sheath assembly comprising the needle array and a main unit comprising the motor assembly.

46. A needling device, comprising:
a plurality of needles forming a needle array; and
a motor assembly for driving the needle array,
wherein each of the plurality of needles comprises a needle tip at one end, and tapers at a taper angle and along a taper length to a maximum needle diameter at the other end, and
wherein the needle tip has a tip radius ranging from approximately 0.015 mm to approximately 0.025 mm.

47. The needling device of claim 46, wherein, in use, a skin reference surface of the needling device is in contact with a subject's skin, and the skin reference surface has a surface area ranging from approximately 45 mm² to approximately 105 mm².

48. The needling device of any one of claim 46 or 47, wherein, in use, a skin reference surface of the needling device is in contact with a subject's skin, and an average distance between each needle of the plurality of needles of the needle array and the skin reference surface area ranges from approximately 0.10 mm to approximately 2.5 mm.

49. The needling device of claim 48, wherein a distance between each needle of the plurality of needles of the needle array and the skin reference surface is the same for all needles.

50. The needling device of claim 48, wherein a first distance between one of the plurality of needles of the needle array and the skin reference surface is different than a second distance between another of the plurality of needles of the needle array and the skin reference surface.

51. The needling device of any one of claims 46 to 50, wherein the motor assembly comprises a motor linkage, and wherein the motor linkage comprises a rotational component with a total mass ranging from approximately 0.5 grams to approximately 35 grams and a rotational radius ranging from approximately 1.5 mm to approximately 3.5 mm, and the motor linkage comprises a linear component with a total mass ranging from approximately 1.5 grams to approximately 3.5 grams.

52. The needling device of any one of claims 46 to 51, wherein an actual penetration depth of the plurality of needles into a subject's skin does not exceed a depth setting of the needling device.

53. The needling device of any one of claims 46 to 52, wherein a mean value of an actual penetration depth of the plurality of needles is at least 0.2 mm in response to a depth setting of the needling device of 0.5 mm, at least 0.6 mm in response to a depth setting of the needling device of 1.5 mm, and at least 0.75 mm in response to a depth setting of the needling device of 2.0 mm.

54. The needling device of any one of claims 46 to 53, wherein, in use, an actual penetration depth of the plurality of needles is at least 50% of a target depth based on a depth setting of the device for at least 45% of all needle strikes of the plurality of needles.

55. The needling device of any one of claims 46 to 54, wherein, in use, an actual penetration depth of the plurality of needles is at least 50% of a target depth based on a depth setting of the device for at least 35% of all needle strikes of the plurality of needles.

56. The needling device of any one of claims 46 to 55, wherein, in use, an actual penetration depth of the plurality of needles is at least 50% of a target depth based on a depth setting of the device for at least 25% of all needle strikes of the plurality of needles.

57. The needling device of any one of claims 46 to 56, wherein, in use, an actual penetration depth of the plurality of needles is at least 50% of a target depth based on a depth setting of the device for at least 15% of all needle strikes of the plurality of needles.

58. The needling device of any one of claims 46 to 57, further comprising a sheath assembly comprising the needle array and a main unit comprising the motor assembly.

59. A needling device, comprising:
a plurality of needles forming a needle array; and
a motor assembly for driving the needle array,
wherein, in use, a skin reference surface of the needling device is in contact with a subject's skin, and the skin reference surface has a surface area ranging from approximately 45 mm² to approximately 105 mm².

60. The needling device of claim 59, wherein, in use, a skin reference surface of the needling device is in contact with a subject's skin, and an average distance between each needle of the plurality of needles of the needle array and the skin reference surface area ranges from approximately 0.10 mm to approximately 2.5 mm.

61. The needling device of claim 60, wherein a distance between each needle of the plurality of needles of the needle array and the skin reference surface is the same for all needles.

62. The needling device of claim 60, wherein a first distance between one of the plurality of needles of the needle array and the skin reference surface is different than a second distance between another of the plurality of needles of the needle array and the skin reference surface.

63. The needling device of any one of claims 59 to 62, wherein the motor assembly comprises a motor linkage, and wherein the motor linkage comprises a rotational component with a total mass ranging from approximately 0.5 grams to approximately 35 grams and a rotational radius ranging from approximately 1.5 mm to approximately 3.5 mm, and the motor linkage comprises a linear component with a total mass ranging from approximately 1.5 grams to approximately 3.5 grams.

64. The needling device of any one of claims 59 to 63, wherein an actual penetration depth of the plurality of needles does not exceed a depth setting of the needling device.

65. The needling device of any one of claims 59 to 64, wherein a mean value of an actual penetration depth of the plurality of needles is at least 0.2 mm in response to a depth setting of the needling device of 0.5 mm, at least 0.6 mm in response to a depth setting of the needling device of 1.5 mm, and at least 0.75 mm in response to a depth setting of the needling device of 2.0 mm.

66. The needling device of any one of claims 59 to 65, wherein, in use, an actual penetration depth of the plurality of needles is at least 50% of a target depth based on a depth setting of the device for at least 45% of all needle strikes of the plurality of needles.

67. The needling device of any one of claims 59 to 66, wherein, in use, an actual penetration depth of the plurality of needles is at least 50% of a target depth based on a depth setting of the device for at least 35% of all needle strikes of the plurality of needles.

68. The needling device of any one of claims 59 to 67, wherein, in use, an actual penetration depth of the plurality of needles is at least 50% of a target depth based on a depth setting of the device for at least 25% of all needle strikes of the plurality of needles.

69. The needling device of any one of claims 59 to 68, wherein, in use, an actual penetration depth of the plurality of needles is at least 50% of a target depth based on a depth setting of the device for at least 15% of all needle strikes of the plurality of needles.

70. The needling device of any one of claims 59 to 69, further comprising a sheath assembly comprising the needle array and a main unit comprising the motor assembly.

71. A needling device, comprising:
a plurality of needles forming a needle array; and
a motor assembly for driving the needle array,
wherein, in use, a skin reference surface of the needling device is in contact with a subject's skin, and an average distance between each needle of the plurality of needles of the needle array and the skin reference surface area ranges from approximately 0.10 mm to approximately 2.5 mm.

72. The needling device of claim 71, wherein a distance between each needle of the plurality of needles of the needle array and the skin reference surface is the same for all needles.

73. The needling device of claim 71, wherein a first distance between one of the plurality of needles of the needle array and the skin reference surface is different than a second distance between another of the plurality of needles of the needle array and the skin reference surface.

74. The needling device of any one of claims 71 to 73, wherein the motor assembly comprises a motor linkage, and wherein the motor linkage comprises a rotational component with a total mass ranging from approximately 0.5 grams to approximately 35 grams and a rotational radius ranging from approximately 1.5 mm to approximately 3.5 mm, and the motor linkage comprises a linear component with a total mass ranging from approximately 1.5 grams to approximately 3.5 grams.

75. The needling device of any one of claims 71 to 74, wherein an actual penetration depth of the plurality of needles into a subject's skin does not exceed a depth setting of the needling device.

76. The needling device of any one of claims 71 to 75, wherein a mean value of an actual penetration depth of the plurality of needles is at least 0.2 mm in response to a depth setting of the needling device of 0.5 mm, at least 0.6 mm in response to a depth setting of the needling device of 1.5 mm, and at least 0.75 mm in response to a depth setting of the needling device of 2.0 mm.

77. The needling device of any one of claims 71 to 76, wherein, in use, an actual penetration depth of the plurality of needles is at least 50% of a target depth based on a depth setting of the device for at least 45% of all needle strikes of the plurality of needles.

78. The needling device of any one of claims 71 to 77, wherein, in use, an actual penetration depth of the plurality of needles is at least 50% of a target depth based on a depth setting of the device for at least 35% of all needle strikes of the plurality of needles.

79. The needling device of any one of claims 71 to 78, wherein, in use, an actual penetration depth of the plurality of needles is at least 50% of a target depth based on a depth setting of the device for at least 25% of all needle strikes of the plurality of needles.

80. The needling device of any one of claims 71 to 79, wherein, in use, an actual penetration depth of the plurality of needles is at least 50% of a target depth based on a depth setting of the device for at least 15% of all needle strikes of the plurality of needles.

81. The needling device of any one of claims 71 to 80, further comprising a sheath assembly comprising the needle array and a main unit comprising the motor assembly.

82. A needling device, comprising:
a plurality of needles forming a needle array; and
a motor assembly for driving the needle array,
wherein the motor assembly comprises a motor linkage, and wherein the motor linkage comprises a rotational component with a total mass ranging from approximately 0.5 grams to approximately 35 grams and a rotational radius ranging from approximately 1.5 mm to approximately 3.5 mm, and the motor linkage comprises a linear component with a total mass ranging from approximately 1.5 grams to approximately 3.5 grams.

83. The needling device of claim 82, wherein an actual penetration depth of the plurality of needles into a subject's skin does not exceed a depth setting of the needling device.

84. The needling device of any one of claim 82 or 83, wherein a mean value of an actual penetration depth of the plurality of needles is at least 0.2 mm in response to a depth setting of the needling device of 0.5 mm, at least 0.6 mm in response to a depth setting of the needling device of 1.5 mm, and at least 0.75 mm in response to a depth setting of the needling device of 2.0 mm.

85. The needling device of any one of claims 82 to 84, wherein, in use, an actual penetration depth of the plurality of needles is at least 50% of a target depth based on a depth setting of the device for at least 45% of all needle strikes of the plurality of needles.

86. The needling device of any one of claims 82 to 85, wherein, in use, an actual penetration depth of the plurality of needles is at least 50% of a target depth based on a depth setting of the device for at least 35% of all needle strikes of the plurality of needles.

87. The needling device of any one of claims 82 to 86, wherein, in use, an actual penetration depth of the plurality of needles is at least 50% of a target depth based on a depth setting of the device for at least 25% of all needle strikes of the plurality of needles.

88. The needling device of any one of claims 82 to 87, wherein, in use, an actual penetration depth of the plurality of needles is at least 50% of a target depth based on a depth setting of the device for at least 15% of all needle strikes of the plurality of needles.

89. The needling device of any one of claims 82 to 88, further comprising a sheath assembly comprising the needle array and a main unit comprising the motor assembly.
